(19) United States
(12) Patent Application Publication
(10) Pub. No.: US 2011/0224221 A1
(43) Pub. Date: Sep. 15, 2011

(54) HEMATOPOIETIC PROTECTION AGAINST IONIZING RADIATION USING SELECTIVE CYCLIN-DEPENDENT KINASE 4/6 INHIBITORS

(76) Inventors: Norman E. Sharpless, Chapel Hill, NC (US); Chad D. Torrice, Chapel Hill, NC (US); Matthew R. Ramsey, Chapel Hill, NC (US); Soren Johnson, Chapel Hill, NC (US); Jessica F. Bell, Chapel Hill, NC (US)

(21) Appl. No.: 13/122,017

(22) PCT Filed: Oct. 1, 2009
(86) PCT No.: PCT/US09/59254

§ 371 Date: May 26, 2011

Related U.S. Application Data
(60) Provisional application No. 61/101,824, filed on Oct. 1, 2008.

(51) Int. Cl. A61K 31/497; A61K 31/519; A61K 31/407; A61P 43/00

(52) U.S. Cl. 514/252.16; 514/264.1; 514/411

(57) ABSTRACT

Methods for reducing or preventing the effects of ionizing radiation in healthy cells arc provided. The methods relate to the use of selective cyclin-dependent kinase (CDK) 4/6 inhibitors to induce transient quiescence in CDK4/6 dependent cells, such as hematopoietic stem cells and/or hematopoietic progenitor cells. Radioprotection can be effected in mammals by treatment with selective CDK4/6 inhibitor compounds either before, at the same time as, or after exposure to the ionizing radiation.

### RELATED APPLICATIONS

The presently disclosed subject matter is based on and claims the benefit of U.S. Provisional Application Ser. No. 61/101,824, filed Oct. 1, 2008; the disclosure of which is incorporated herein by reference in its entirety.

### GOVERNMENT INTEREST

This presently disclosed subject matter was made with U.S. Government support under Grant No. RO1 AG024379-01 and K08 CA90679 awarded by the National Institutes of Health through the National Institute on Aging and the National Cancer Institute. Thus, the U.S. Government has certain rights in the presently disclosed subject matter.

### TECHNICAL FIELD

The presently disclosed subject matter relates to methods of protecting healthy cells from damage due to ionizing radiation. In particular, the presently disclosed subject matter relates to the radioprotective action of selective cyclin dependent kinase 4/6 (CDK4/6) inhibitors administered to subjects that have been exposed to, will be exposed to, or that are at risk of exposure to ionizing radiation.

### ABBREVIATIONS

° C.=degrees Celsius
%=percentage
μL=microliters
μM=micromolar
2BrIC=2-bromo-12,13-dihydro-5H-indolo[2,3-a]pyrrolo[3,4]-carbazole-5,6-dione
BM=bone marrow
BM-MNC=bone marrow mononuclear cells
BrdU=5-bromo-2-deoxyuridine
CAFC=cobblestone area-forming cell
CBC=complete blood count
CDK=cyclin-dependent kinase
CDK4/6=cyclin dependent kinase 4 and/or cyclin-dependent kinase 6
CLP=common lymphoid progenitors
CMP=common myeloid progenitors
DMF=dimethylformamide
DMSO=dimethyl sulfoxide
DNA=deoxyribonucleic acid
ESI=electrospray ionization
EtOAc=ethyl acetate
EtOH=ethanol
FBS=fetal bovine serum
g=gram
G-CSF=granulocyte colony stimulating factor
GEMM=genetically engineered murine model
GM-CSF=granulocyte-macrophage colony stimulating factor
GMP=granulocyte-monocyte progenitors
Gy=gray
h=hours
HSC=hematopoietic stem cells
HSPC=hematopoietic stem and progenitor cells
IC₅₀=50% inhibitory concentration
i.p.=intraperitoneal
IR=ionizing radiation
kg=kilogram
LC-MS=liquid chromatography-mass spectroscopy
LD₉₀=90% lethal dose
LT-HSC=long term hematopoietic stem cell
M=molar
MEP=megakaryocyte-erythroid progenitors
mg=milligrams
MHz=megaHertz
mL=milliliters
mmol=millimoles
mol=moles
Mp=melting point
MPP=multipotent progenitor
NBS=N-bromosuccinimide
nM=nanomolar
NMR=nuclear magnetic resonance
PBS=phosphate buffered saline
PD=6-acetyl-8-cyclopentyl-5-methyl-2-(5-piperazin-1-yl-pyridin-2-ylamino)-8H-pyrido-[2,3-d]-pyrimidin-7-one (also referred to as PD 0332991)
PQ=pharmacologic quiescence
RB=retinoblastoma tumor suppressor protein
r.t.=room temperature
SEM=standard error of the mean
ST-HSC=short term hematopoietic stem cell
Sv=Sievert
TBI=total body irradiation
tHDF=telomerized human diploid fibroblast
THF=tetrahydrofuran
UV=ultraviolet

### BACKGROUND

Ionizing radiation (IR) has an adverse effect on cells and tissues, primarily through cytotoxic effects. In humans, exposure to ionizing radiation occurs primarily through therapeutic techniques (such as anticancer radiotherapy) or through occupational and environmental exposure.

A major source of exposure to ionizing radiation is the administration of therapeutic radiation in the treatment of cancer or other proliferative disorders. Subjects exposed to therapeutic doses of ionizing radiation typically receive between 0.1 and 2 gray (Gy) per treatment, and can receive as high as 5 Gy per treatment. Depending on the course of treatment prescribed by the treating physician, multiple doses can be received by a subject over the course of several weeks to several months.

Therapeutic radiation is generally applied to a defined area of the subject's body which contains abnormal proliferative tissue, in order to maximize the dose absorbed by the abnormal tissue and minimize the dose absorbed by the nearby normal tissue. However, it is difficult (if not impossible) to selectively administer therapeutic ionizing radiation to the abnormal tissue. Thus, normal tissue proximate to the abnormal tissue is also exposed to potentially damaging doses of ionizing radiation throughout the course of treatment. There are also some treatments that require exposure of the subject's entire body to the radiation, in a procedure called “total body irradiation” (i.e., TBI). The efficacy of radiotherapeutic techniques in destroying abnormal proliferative cells is therefore balanced by associated cytotoxic effects on nearby normal cells. Because of this, radiotherapy techniques have an inherently narrow therapeutic index which results in the inadequate treatment of most tumors. Even the best radiotherapeutic techniques can result in incomplete tumor reduction, tumor recurrence, increasing tumor burden, and induction of radiation resistant tumors.

Numerous methods have been designed to reduce normal tissue damage while still delivering effective therapeutic doses of ionizing radiation. These techniques include brachytherapy, fractionated and hyperfractionated dosing, complicated dose scheduling and delivery systems, and high voltage therapy with a linear accelerator. However, such techniques only attempt to strike a balance between the therapeutic and undesirable effects of the radiation, and full efficacy has not been achieved.

Exposure to ionizing radiation can also occur in the occupational/industrial setting. Occupational doses of ionizing radiation can be received by persons whose job involves exposure (or potential exposure) to radiation, for example in the nuclear power and nuclear weapons industries. Incidents such as the 1979 accident at Three Mile Island nuclear power plant, which released radioactive material into the reactor containment building and surrounding environment, illustrate the potential for harmful exposure. Even in the absence of catastrophic events, workers in the nuclear power industry are subject to higher levels of radiation than the general public.

Military personnel stationed on vessels powered by nuclear reactors, or soldiers required to operate in areas contaminated by radioactive fallout, risk similar exposure to ionizing radiation. Military personnel can also be subjected to ionizing radiation as the result of encountering radiological devices on the battlefield. Occupational exposure can also occur in salvage, rescue, and emergency personnel called in to deal with catastrophic events involving a nuclear reactor or radioactive material. Occupational exposure can further affect astronauts during space travel in the absence of adequate radiation shielding. Other sources of occupational exposure can be from machine parts, plastics, and solvents left over from the manufacture of radioactive medical products, smoke alarms, emergency signs, and other consumer goods.

Even in the absence of occupational risks, humans (and other animals, such as livestock and pets) can be exposed to ionizing radiation from the environment. The primary source of exposure to significant amounts of environmental radiation is from nuclear power plant accidents, such as those at Three Mile Island, Chernobyl and Tokaimura. A 1982 study by Sandia National Laboratories estimated that a “worst-case” nuclear accident could result in a death toll of more than 100,000 and long-term radioactive contamination of large areas of land. Humans and other animals can further be subjected to ionizing radiation as the result of radiological warfare or terrorist attack.

Radiation exposure from any source can be classified as acute (a single large exposure) or chronic (a series of small low-level, or continuous low-level exposures spread over time). Radiation sickness generally results from an acute exposure of a sufficient dose, and presents with a characteristic set of symptoms that appear in an orderly fashion, including hair loss, weakness, vomiting, diarrhea, skin burns and bleeding from the gastrointestinal tract and mucous membranes. Genetic defects, sterility and cancers (particularly bone marrow cancer) often develop over time. Chronic exposure is usually associated with delayed medical problems such as cancer and premature aging.

Generally, an acute exposure of over 200,000 millirem leads to death while lower dosages cause radiation sickness. Acute doses of up to about 7 Gy can lead to an effect known as “hematologic syndrome” (i.e., IR-induced bone marrow suppression). Acute doses higher than 7 Gy can lead to effects known as “gastrointestinal syndrome” or (in the cases of the most severe exposure) “cardiovasucular/central nervous system syndrome.” Even lower acute doses (for example, an acute total body radiation dose of 100,000-125,000 millirem (equivalent to 1 Gy) received in less than one week) can result in observable physiologic effects such as skin burns or rashes, mucosal and gastro-intestinal bleeding, nausea, diarrhea and/or excessive fatigue. Longer term cytotoxic and genetic effects such as hematopoietic and immune cell destruction, hair loss (alopecia), gastrointestinal and oral mucosal sloughing, venoocclusive disease of the liver and chronic vascular hyperplasia of cerebral vessels, cataracts, pneumonites, skin changes, and an increased incidence of cancer can also manifest over time. Acute doses of less than 10,000 millirem (equivalent to 0.1 Gy) typically do not result in immediately observable biologic or physiologic effects, although long term cytotoxic or genetic effects can occur.

While anti-radiation suits or other protective gear can be effective at reducing radiation exposure, such gear is expensive, unwieldy, and generally not available to public. Moreover, radioprotective gear will not protect normal tissue adjacent to a tumor from stray radiation exposure during radiotherapy. Nor can radioprotective gear help subjects who have already incurred unexpected radiation exposure.

Several methods have been proposed to provide ‘radioprotection’ (treatment to protect against undesired effects of IR given prior to IR exposure) or ‘radiomitigation’ (i.e., treatment to protect from undesired effects of IR given after IR exposure). See Weiss and Landauer, Int. J. Radiat. Biol., 85, 539-573 (2009). Amifostine, an oxygen radical scavenger, provides protection to clinical radiation-induced mucositis, but is not effective at reducing hematologic toxicity. Additional radioprotectant therapies, particularly with regard to IR associated anemia and neutropenia, include the use of growth factors. Hematopoietic growth factors are available on the market as recombinant proteins. These proteins include granulocyte colony stimulating factor (G-CSF) and granulocyte-macrophage colony stimulating factor (GM-CSF) and their derivatives for the treatment of neutropenia, and erythropoietin (EPO) and its derivatives for the treatment of anemia. However, these recombinant proteins are expensive. Moreover, EPO has significant toxicity in cancer patients, leading to increased thrombosis, relapse and death in several large randomized trials. G-CSF and GM-CSF can increase the late (>2 years post-therapy) risk of secondary bone marrow disorders such as leukemia and myelodysplasia. Consequently, their use is restricted and not readily available to all patients in need. Further, while growth factors can hasten recovery of some blood cell lineages, no therapy exists to treat suppression of platelets, macrophages, T-cells or B-cells. Chelating agents and iodine supplementation can mitigate the toxicities of specific radioactive isotopes, but are not effective at mitigating the hematologic toxicity of IR.

The non-selective kinase inhibitor staurosporine has been shown to afford protection from DNA damaging agents in some cultured cell types. See Chen et al., J. Natl. Cancer Inst., 92, 1999-2008 (2000); and Ojeda et al., Int J. Radiat. Biol., 61, 663-667 (1992). Staurosporine is a naturally occurring product and non-selective kinase inhibitor that binds most mammalian kinases with high affinity. See Karaman et al., Nat. Biotechnol., 26, 127-132 (2008). Staurosporine treatment can elicit an array of cellular responses including apoptosis, cell cycle arrest and cell cycle checkpoint compromise depending on cell type, drug concentration, and length of exposure. For example, staurosporine has been shown to sensitize cells to DNA damaging agents such as ionizing radiation and chemotherapy (see Bernhard et al., Int. J. Radiat. Biol., 69, 575-584 (1996); Teyssier et al., Bull. Cancer, 86, 345-357 (1999); Hallahan et al., Radiat. Res., 129, 345-350 (1992); Zhanq et al., J. Neurooncol., 15, 1-7 (1993); Guo et al., Int. J. Radiat. Biol., 82, 97-109 (2006); Bucher and Britten, Br. J. Cancer, 98, 523-528 (2008); Laredo et al., Blood, 84, 229-237 (1994); Luo et al., Neoplasia, 3, 411-419 (2001); Wang et al., Yao Xue Xue Bao, 31, 411-415 (1996); Chen et al., J. Natl. Cancer Inst., 92, 1999-2008 (2000); and Hirose et al., Cancer Res., 61, 5843-5849 (2001)) through several claimed mechanisms including abrogation of a G2 checkpoint response. The mechanism whereby staurosporine treatment affords protection from DNA damaging agents in some cultured cell types is unclear, with a few possible mechanisms suggested including inhibition of protein kinase C or decreasing CDK4 protein levels. See Chen et al., J. Natl. Cancer Inst., 92, 1999-2008 (2000); and Ojeda et al., Int J. Radiat. Biol., 61, 663-667 (1992). No effect of staurosporine has been shown on hematopoietic progenitors, nor has staurosporine use well after exposure to DNA damaging agents been shown to afford protection. Staurosporine's non-selective kinase inhibition has led to significant toxicities independent of its effects on the cell cycle (e.g. hyperglycemia) after in vivo administration to mammals and these toxicities have precluded its clinical use.

Accordingly, there is an ongoing need for practical methods to protect subjects who are scheduled to incur, are at risk for incurring, or who have already incurred, exposure to ionizing radiation. In the context of therapeutic irradiation, it is desirable to enhance protection of normal cells while causing tumor cells to remain vulnerable to the detrimental effects of the radiation. Furthermore, it is desirable to provide systemic protection from anticipated or inadvertent total body irradiation, such as can occur with occupational or environmental exposures, or with certain therapeutic techniques.

### SUMMARY

The presently disclosed subject matter provides a method of reducing or preventing the effects of ionizing radiation on healthy cells in a subject who has been exposed to, will be exposed to, or is at risk of incurring exposure to ionizing radiation, wherein said healthy cells are hematopoietic stem cells or hematopoietic progenitor cells, the method comprising administering to the subject an effective amount of an inhibitor compound, or a pharmaceutically acceptable form thereof, wherein the inhibitor compound selectively inhibits cyclin-dependent kinase 4 (CDK4) and/or cyclin-dependent kinase 6 (CDK6).

In some embodiments, the inhibitor compound is selected from the group consisting of a pyrido[2,3-d]pyrimidine, a triaminopyrimidine, an aryl[a]pyrrolo[3,4-c]carbazole, a nitrogen-containing heteroaryl-substituted urea, a 5-pyrimidinyl-2-aminothiazole, a benzothiadiazine, and an acridinethione. In some embodiments, the pyrido[2,3-d]pyrimidine is a pyrido[2,3-d]pyrimidin-7-one or a 2-amino-6-cyano-pyrido[2,3-d]pyrimidin-4-one. In some embodiments, the pyrido[2,3-d]pyrimidin-7-one is a 2-(2′-pyridyl)amino pyrido[2,3-d]pyrimidin-7-one. In some embodiments, the pyrido[2,3-d]pyrimidin-7-one is 6-acetyl-8-cyclopentyl-5-methyl-2-(5-piperazin-1-yl-pyridin-2-ylamino)-8H-pyrido-[2,3-d]pyrimidin-7-one.

In some embodiments, the aryl[a]pyrrolo[3,4-c]carbazole is selected from the group consisting of a napthyl[a]pyrrolo[3,4-c]carbazole, an indolo[a]pyrrolo[3,4-c]carbazole, a quinolinyl[a]pyrrolo[3,4-c]carbazole, and an isoquinolinyl[a]pyrrolo[3,4-c]carbazole. In some embodiments, the aryl[a]pyrrolo[3,4-c]carbazole is 2-bromo-12,13-dihydro-5H-indolo[2,3-a]pyrrolo[3,4]-carbazole-5,6-dione.

In some embodiments, the inhibitor compound selectively inhibits both CDK4 and CDK6. In some embodiments, the inhibitor compound is a non-naturally occurring compound.

In some embodiments, the inhibitor compound selectively induces G1 arrest in CDK4- and/or CDK6-dependent cells. In some embodiments, the inhibitor compound induces substantially pure G1 arrest in CDK4- and/or CDK6-dependent cells.

In some embodiments, the inhibitor compound is substantially free of off-target effects. In some embodiments, the off-target effects are one or more of the group consisting of long term toxicity, anti-oxidant effects, estrogenic effects, tyrosine kinase inhibition, inhibition of cyclin-dependent kinases (CDKs) other than cyclin-dependent kinase 4/6 (CDK4/6), and cell cycle arrest in CDK4/6-independent cells.

In some embodiments, the subject is a mammal. In some embodiments, the inhibitor compound is administered to the subject by one of the group consisting of oral administration, topical administration, intranasal administration, inhalation, and intravenous administration.

In some embodiments, the inhibitor compound is administered to the subject prior to exposure to the ionizing radiation, during exposure to the ionizing radiation, after exposure to the ionizing radiation, or a combination thereof. In some embodiments, the inhibitor compound is administered to the subject less than about 24 hours prior to exposure to the ionizing radiation. In some embodiments, the inhibitor compound is administered to the subject prior to exposure to the ionizing radiation such that the compound reaches peak serum levels during exposure to the ionizing radiation. In some embodiments, the inhibitor compound is 6-acetyl-8-cyclopentyl-5-methyl-2-(5-piperazin-1-yl-pyridin-2-ylamino)-8H-pyrido-[2,3-d]pyrimidin-7-one and said inhibitor compound is administered orally to the subject 4 hours prior to exposure to the ionizing radiation.

In some embodiments, the inhibitor compound is administered to the subject after exposure to the ionizing radiation. In some embodiments, the inhibitor compound is administered to the subject about 24 hours or more after exposure to the ionizing radiation.

In some embodiments, the healthy cells are selected from the group consisting of long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs) and megakaryocyte-erythroid progenitors (MEPs). In some embodiments, administration of the inhibitor compound provides temporary pharmacologic quiescence of hematopoietic stem and/or progenitor cells in the subject.

In some embodiments, the subject has incurred ionizing radiation or is at risk of incurring exposure to ionizing radiation as the result of radiological agent exposure during warfare, a radiological terrorist attack, an industrial accident, other occupational exposure or space travel.

In some embodiments, the subject is undergoing radio-therapy to treat a disease. In some embodiments, administration of the inhibitor compound does not affect growth of diseased cells. In some embodiments, the disease is cancer. In some embodiments, the cancer is characterized by one or more of the group consisting of increased activity of cyclin-dependent kinase 1 (CDK1), increased activity of cyclin-dependent kinase 2 (CDK2), loss or absence of retinoblastoma tumor suppressor protein (RB), high levels of MYC expression, increased cyclin E and increased cyclin A. In some embodiments, administration of the inhibitor compound allows for a higher dose of ionizing radiation to be used to treat the disease than the dose that would be used in the absence of administration of the inhibitor compound.

In some embodiments, the method is free of long-term hematologic toxicity. In some embodiments, administration of the inhibitor compound results in reduced anemia, reduced lymphopenia, reduced thrombocytopenia, or reduced neutropenia compared to that expected after exposure to ionizing radiation in the absence of administration of the inhibitor compound.

It is an object of the presently disclosed subject matter to provide methods of protecting healthy cells in subjects from the effects of ionizing radiation by administering to the subject an effective amount of a selective CDK4 and/or CDK6 inhibitor.

An object of the presently disclosed subject matter having been stated hereinabove, and which is achieved in whole or in part by the presently disclosed subject matter, other objects will become evident as the description proceeds when taken in connection with the accompanying drawings as best described herein below.

### BRIEF DESCRIPTION OF THE DRAWINGS

FIG. 1 is a schematic drawing of hematopoiesis, the hierarchical proliferation of hematopoietic stem cells (HSC) and progenitor cells with increasing differentiation upon proliferation.

FIG. 2A is a series of representative grayscale images of the progression of an autochthonous TyrRAS+Ink4a/Arf−/−melanoma despite daily oral therapy with 6-acetyl-8-cyclopentyl-5-methyl-2-(5-piperazin-1-yl-pyridin-2-ylamino)-8H-pyrido-[2,3-d]-pyrimidin-7-one (PD 0332991) showing that a p16INK4-deficient genetically engineered murine model (GEMM) of melanoma is insensitive to selective cyclin-dependent kinase 4/6 (CDK4/6) inhibition.

FIG. 2B is a graph showing tumor growth in matched treated and untreated cohorts after 16 consecutive days of 6-acetyl-8-cyclopentyl-5-methyl-2-(5-piperazin-1-yl-pyridin-2-ylamino)-8H-pyrido-[2,3-d]-pyrimidin-7-one (PD 0332991) treatment at 150 mg/kg/dose once per day. The data show normalized tumor size for 6 tumors in 5 untreated mice (closed triangles) and 7 tumors in 5 treated mice (open circles) over time. Arrows indicate time points where mice were sacrificed for tumor progression morbidity (open arrows for untreated animals; shaded arrows for treated animals). Error bars are +/− the standard error of the mean (SEM).

FIG. 2C is a set of graphs showing the dose-response curves for cell cycle analysis in murine (KPTR1, KPTR4 and KPTR5 from TyrRAS+Ink4a/Arf−/−mice) melanoma cell lines. Cells were treated for 24 hours with 6-acetyl-8-cyclopentyl-5-methyl-2-(5-piperazin-1-yl-pyridin-2-ylamino)-8H-pyrido-[2,3-d]-pyrimidin-7-one (PD0332991) at the dosages indicated in the x-axis prior to 15 minutes of 5-bromo-2-deoxyuridine (BrdU) pulse, cell harvesting, fixation, staining and analysis by flow cytometry. The percentage of cells in the G1 phase is indicated by the data shown in the closed squares. The percentage of cells in the S phase is shown by the data in the open triangles. The percentage of cells in the G2/M phase is indicated by the data in the open circles. The percentage of cells actively proliferating as marked by Ki67 positive staining is shown in closed diamonds. Error bars are +/− the standard error of the mean (SEM).

FIG. 2D is a graph of tumor growth by treatment group. Tumor growth with (open squares) or without (closed diamonds) a single dose of 6-acetyl-8-cyclopentyl-5-methyl-2-(5-piperazin-1-yl-pyridin-2-ylamino)-8H-pyrido-[2,3-d]-pyrimidin-7-one (PD0332991) administered 4 hours prior to 7.5 gray (Gy) total body irradiation (TBI). Closed triangles represent unirradiated, untreated tumors for comparison. n.s. is non-significant for all comparisons between groups receiving 7.5 Gy TBI. Error bars indicate standard error of the mean. Cyclin-dependent kinase 4/6 (CDK4/6) inhibitor treatment does not decrease that anti-tumor effect of therapeutic radiotherapy.

FIG. 2E is a set of Kaplan-Meyer survival curves showing overall mortality, tumor-specific mortality and mortality to radiation toxicity of tumor bearing mice treated with 7.5 gray (Gy) of total body irradiation (TBI) with (solid line, n=8) or without (dashed line n=11) 6-acetyl-8-cyclopentyl-5-methyl-2-(5-piperazin-1-yl-pyridin-2-ylamino)-8H-pyrido-[2,3-d]-pyrimidin-7-one (PD 0332991) treatment 4 hours prior to ionizing radiation (IR). P-values calculated using the log-rank test. Cyclin-dependent kinase 4/6 (CDK4/6) inhibitor treatment does not appear to increase tumor-related mortality, but does appear to provide protection from radiation toxicity.

FIG. 3A is a set of graphs showing the dose-response curves for cycle analysis in both cyclin-dependent kinase 4/6 (CKD4/6)-dependent cells (human telomerized human diploid fibroblasts (tHDFs, left-hand column of graphs) and a CDK4/6-dependent human melanoma cell line (WM2664, middle column of graphs)) and in CDK4/6-independent cells (a human retinoblastoma tumor suppressor protein (RB)-null melanoma cell line (A2058), right-hand column of graphs). Cells were treated for 24 hours with a selective or nonselective CDK4/6 inhibitor compound (from top to bottom: flavopiridol; R547 (compound 7); roscovitine; 2-bromo-12,13-dihydro-5H-indolo[2,3-a]pyrrolo[3,4]carbazole-5,6-dione (2BrIC); and 6-acetyl-8-cyclopentyl-5-methyl-2-(5-piperazin-1-yl-pyridin-2-ylamino)-8H-pyrido-[2,3-d]-pyrimidin-7-one (PD0332991)) at the dosages indicated in the x-axis prior to 15 minutes of 5-bromo-2-deoxyuridine (BrdU) pulse, cell harvesting, fixation, staining and analysis by flow cytometry. The percentage of cells in the G1 phase is indicated by the data shown in the open triangles, while the percentage of cells in the S phase is shown by the data in the shaded circles and the percentage of cells in the G2/M phase is indicated by the data in the double “x”s.

FIG. 3B is a set of representative cell cycle dot-plots corresponding to the data shown in the graphs described for FIG. 3A. Also shown (top row) are representative cell cycle dot-plots for cells that were not treated with a specific or nonspecific CDK inhibitor, but only with dimethyl sulfoxide (DMSO) as a control. Increasing DNA content is shown on the x-axis, measured by propidium iodide staining, while 5-bromo-2-deoxyuridine (BrdU) uptake is shown on the y-axis.

FIG. 4A are images of Western blots of a DNA damage response marker (phospho-P53) in telomerized human diploid fibroblast (tHDF) cell lysates 3 hours after or 6 hours after a 6 gray (Gy) dose of ionizing radiation (IR) or following no IR, as indicated above the blot images. Prior to IR, the tHDFs were either treated (+) or untreated (−) with 6-acetyl-8-cyclopentyl-5-methyl-2-(5-piperazin-1-yl-pyridin-2-ylamino)-8H-pyrido-[2,3-d]-pyrimidin-7-one (PD0332991) at 100 nM for 24 hours. Actin western blot is shown as a loading control.

FIG. 4B is a bar graph showing the normalized phospho-P53 intensity in the telomerized human diploid fibroblast (tHDF) cells described in FIG. 4A. Data from cells treated with dimethyl sulfoxide (DMSO) as a control is shown in the open bars, data from the 6-acetyl-8-cyclopentyl-5-methyl-2-(5-piperazin-1-yl-pyridin-2-ylamino)-8H-pyrido-[2,3-d]-pyrimidin-7-one (PD0332991)-treated cells is shown in the shaded bars.

FIG. 5A is a set of grayscale 40× images of phospho-γH2AX foci (nuclear) and phalloidin staining (cytoplasmic) of telomerized human diploid fibroblasts (tHDF) with (bottom row) and without (top row) 6 Gy ionizing radiation (IR). Indicated cultures were treated for 24 hours with 100 nM 6-acetyl-8-cyclopentyl-5-methyl-2-(5-piperazin-1-yl-pyridin-2-ylamino)-8H-pyrido-[2,3-d]-pyrimidin-7-one (PD 0332991; right-hand column) or vehicle only (dimethyl sulfoxide, left-hand row) prior to IR exposure. Scale bar=50 μm.

FIG. 5B is a graph showing the quantification of mean nuclear fluorescent intensity from γH2AX immunofluorescence images with and without 6 gray (Gy) ionizing radiation (IR) at 0 and 3 hours after exposure with 6-acetyl-8-cyclopentyl-5-methyl-2-(5-piperazin-1-yl-pyridin-2-ylamino)-8H-pyrido-[2,3-d]-pyrimidin-7-one (PD0332991) as in FIG. 5A. N=139 or greater for each condition; box=middle 50%, whiskers=0-25% and 75-100%. Significance determined by Kruskal-Wallis with Dunn post-hoc test for pairwise comparisons (*** p<0.0001).

FIG. 5C is a pair of representative images at 20× magnification from the comet tail assay, a direct measure of DNA damage, performed on cells treated with 8 gray (Gy) ionizing radiation (IR) with (right-hand image) or without (left-hand image) pre-treatment with the selective cyclin-dependent kinase 4/6 (CDK4/6) inhibitor 6-acetyl-8-cyclopentyl-5-methyl-2-(5-piperazin-1-yl-pyridin-2-ylamino)-8H-pyrido-[2,3-d]-pyrimidin-7-one (PD0332991). Telomerized human diploid fibroblasts were irradiated and then immobilized. After immobilization, cell nuclei were exposed to an electric field by gel electrophoresis, with fragmented DNA migrating further than undamaged DNA. Treatment with 8 Gy IR induces significant DNA fragmentation, which is greatly attenuated by treatment with selective CDK4/6 inhibitors.

FIG. 5D is a bar graph quantifying results of the comet tail assay as described in FIG. 5C for the indicated doses (0, 3, 4, 6, or 8 gray (Gy)) of ionizing radiation after treatment with 6-acetyl-8-cyclopentyl-5-methyl-2-(5-piperazin-1-yl-pyridin-2-ylamino)-8H-pyrido-[2,3-d]-pyrimidin-7-one (PD0332991, shaded bars) or vehicle only (dimethyl sulfoxide (DMSO); unshaded bars) at 20× magnification. Selective cyclin-dependent kinase 4/6 (CDK4/6) inhibitors potently reduce comet tail formation, a direct measure of DNA damage, compared to vehicle treatment only. Error bars represent standard error of the mean.

FIG. 5E is a bar graph quantifying results of a comet tail assay similar to that described in FIG. 5C for the indicated doses (0, 2, 4, 6, or 8 gray (Gy)) of ionizing radiation after treatment with 2-bromo-12,13-dihydro-5H-indolo[2,3-a]pyrrolo[3,4]carbazole-5,6-dione (2BrIC; shaded bars) or vehicle only (dimethyl sulfoxide (DMSO), unshaded bars) at 10× magnification. Selective cyclin-dependent kinase 4/6 (CDK4/6) inhibitors potently reduce comet tail formation, a direct measure of DNA damage, compared to vehicle treatment only. Error bars represent standard error of the mean.

FIG. 5F is a set of representative phospho-γH2AX (x-axis) dot plots after 0, 2, 4, 6, or 8 gray (Gy) of ionizing radiation with (right-hand column) or without (left-hand column) treatment with the selective cyclin-dependent kinase 4/6 (CDK4/6) inhibitor 2-bromo-12,13-dihydro-5H-indolo[2,3-a]pyrrolo[3,4]carbazole-5,6-dione (2BrIC) at 2 μM for 24 hours prior to exposure to ionizing radiation (IR). Cells that were not treated with 2BrIC were instead treated with vehicle (dimethyl sulfoxide (DMSO)) only. An increased fraction of phospho-γH2AX (x-axis) expressing cells is noted with increased doses of IR in DMSO treated cells. Treatment with 2BrIC potently decreases IR-induced DNA damage.

FIG. 5G is a bar graph quantifying the results shown in FIG. 5E. Exposure to the selective cyclin-dependent kinase 4/6 (CDK4/6) inhibitor 2-bromo-12,13-dihydro-5H-indolo[2,3-a]pyrrolo[3,4]carbazole-5,6-dione (2BrIC) at 2 μM for 24 hours prior to exposure to ionizing radiation decreases the induction of phospho-γH2AX expression, a marker of DNA damage. Data from the cells treated with 2BrIC is shown in the striped bars; data from cells treated with dimethyl sulfoxide (DMSO) is shown in the stippled bars.

FIG. 6A is a set of micrograph images of crystal violet stained cell cultures of cyclin-dependent kinase 4/6 (CDK4/6)-dependent cells (HS68) plated at different cell/well ratios and treated with either dimethyl sulfoxide (DMSO) as a negative control or with the selective CDK4/6 inhibitor 2-bromo-12,13-dihydro-5H-indolo[2,3-a]pyrrolo[3,4]carbazole-5,6-dione (2BrIC) at 2 μM for 24 hours prior to exposure to ionizing radiation at 0, 1.5, 3, 6, or 9 gray (Gy), as indicated.

FIG. 6B is a graph showing enhanced cell survival in the irradiated HS68 cells treated with 2BrIC as described in FIG. 6A. Data for cells treated with 2-bromo-12,13-dihydro-5H-indolo[2,3-a]pyrrolo[3,4]carbazole-5,6-dione (2BrIC) relative to data for cells treated only with dimethyl sulfoxide (DMSO). Data is plotted as the area/cell ratio for 2BrIC treated cells to dimethyl sulfoxide (DMSO) treated cells. Error bars show standard error of the mean.

FIG. 7 is a graph showing enhanced cell survival in cyclin-dependent kinase 4/6 (CDK4/6)-dependent cells (telomerized human diploid fibroblast cells (tHDFs); shaded diamonds) and not in CDK4/6-independent cells (retinoblastoma tumor suppressor protein (RB)-null melanoma cell line A2058; unshaded diamonds) after pretreatment with 100 nM 6-acetyl-8-cyclopentyl-5-methyl-2-(5-piperazin-1-yl-pyridin-2-ylamino)-8H-pyrido-[2,3-d]-pyrimidin-7-one (PD0332991) and varying doses of ionizing radiation (IR; 0-9 gray (Gy)). Data plotted is the area/cell ratio of PD0332991 treated cells to dimethyl sulfoxide (DMSO) treated cells. Error bars show standard error of the mean.

FIG. 8 is a set of representative cell cycle dot-plots for cyclin-dependent kinase 4/6 (CDK4/6)-dependent (WM2664) human melanoma cells (top two rows) and CDK4/6-independent (A2058) human melanoma cells (bottom two rows) that had been treated with dimethyl sulfoxide (DMSO) as a control or with 300 nM, 1 μM, 3 μM, 10 μM or 30 μM of trans-4-[[6-ethylamino)-2-[[2-phenylmethyl)-1H-indol-5-yl]amino]-4-pyrimidinyl]amino]]-cyclohexanol (CINK4).

FIG. 9A is a set of micrograph images of cell cultures of cyclin-dependent kinase 4/6 (CDK4/6)-dependent cells (HS68) plated at different cell/well ratios and pretreated with either dimethyl sulfoxide (DMSO) as a control or with the nonselective CDK4/6 inhibitor trans-4-[[6-ethylamino)-2-[[1-phenylmethyl)-1H-indol-5-yl]amino]-4-pyrimidinyl]amino]]-cyclohexanol (CINK4) at 6 μM for 24 hours prior to exposure to ionizing radiation at 0, 1.5, 3, 6, or 9 gray (Gy), as indicated. The plates were stained with crystal violet to visualize cell colonies.

FIG. 9B is a graph showing lack of enhanced cell survival in the irradiated HS68 cells pretreated with trans-4-[[6-ethylamino)-2-[[1-phenylmethyl)-1H-indol-5-yl]amino]-4-pyrimidinyl]amino]]cyclohexanol (CINK4) as described in FIG. 9A. Shown in the shaded diamonds is the area/cell ratio of cells treated with CINK4 relative to cells treated with dimethyl sulfoxide (DMSO). Error bars show standard error of the mean.

FIG. 10A is a series of flow cytometry gating schemes for hematopoietic stem cells (HSC, CD150+Lin−Kit+Sca+) and multipotent progenitor (MPP, Lin−Kit+Sca+) cells (top) and myeloid progenitors (Lin−Kit+Sca−, bottom) using cell surface antigens.

FIG. 10B is a series of representative contour plots of proliferation in hematopoietic stem and progenitor cell populations measured by 5-bromo-2-deoxyuridine (BrdU) incorporation and Ki67 expression after 48 hours of no treatment (N=6) or of treatment with 6-acetyl-8-cyclopentyl-5-methyl-2-(5-piperazin-1-yl-pyridin-2-ylamino)-8H-pyrido-[2,3-d]pyrimidin-7-one (PD 0332991). For the last 24 hours of treatment mice were injected with 1 mg 5-bromo-2-deoxyuridine (BrdU) every 6 hours to label proliferating cells. Contours represent 5% density. BrdU incorporation is a measure of G1 to S-phase cell cycle traversal and Ki67 expression is a marker of cycling cells. PD treatment clearly reduces proliferation in these early HSPC.

FIG. 10C is a set of bar graphs showing the quantification of the 5-bromo-2-deoxyuridine (BrdU) incorporation and Ki67 expression data in the untreated (open bars) and treated (shaded bars) cell populations from FIG. 10B. *p,0.05; **p<0.01, ***p<0.001. Error bars show standard error of the mean.

FIG. 10D is a set of bar graphs showing the relative frequencies of Lin−, hematopoietic stem cell (HSC), multi-potent progenitor (MPP) or Lin−cKit+Sca1− populations in 6-acetyl-8-cyclopentyl-5-methyl-2-(5-piperazin-1-yl-pyridin-2-ylamino)-8H-pyrido-[2,3-d]pyrimidin-7-one (PD0332991) untreated (open bars) and treated (shaded bars) cell populations after 48 hours of treatment/no treatment and 24 hours of 5-bromo-2-deoxyuridine (BrdU) exposure, as in FIG. 10B. *p,0.05; **p<0.01, ***p<0.001. Error bars show standard error of the mean. A relative enrichment of HSC and MPP occurs with CDK4/6 inhibitor treatment because more differentiated myeloid cells, which are considerably more abundant, continue to divide and differentiate in the presence of CDK4/6 inhibitor.

FIG. 11A is a series of flow cytometry gating schemes for untreated multipotent progenitor (MPP) cells (top) and 2-bromo-12,13-dihydro-5H-indolo[2,3-a]pyrrolo[3,4]-carbazole-5,6-dione (2BrIC)-treated MPP cells (bottom) using cell surface antigens. In addition to treatment or non-treatment with 2BrIC for 24 hours, cells were also in the presence of 5-bromo-2-deoxyuridine (BrdU).

FIG. 11B is a bar graph showing the percentage of 5-bromo-2-deoxyuridine (BrdU) positive cells in the Lin−Kit+Sca−1 positive untreated and 2-bromo-12,13-dihydro-5H-indolo[2,3-a]pyrrolo[3,4]-carbazole-5,6-dione (2BrIC)-treated cell populations from FIG. 11A. BrdU incorporation is a measure of G1 to S-phase cell cycle traversal, with in vivo 2BrIC treatment clearly reducing proliferation of the MPP.

FIG. 12A is a bar graph showing the effects of 48 hour treatment with 6-acetyl-8-cyclopentyl-5-methyl-2-(5-piperazin-1-yl-pyridin-2-ylamino)-8H-pyrido-[2,3-d]pyrimidin-7-one (PD0332991) on total bone marrow cellularity. The number of bone marrow-mononuclear cells (BM-MNCs) following treatment with PD0332991 for 48 hours are shown by the shaded bar, the number of BM-MNCs following no PD0332991 treatment is shown by the unshaded bar. Error bars show standard error of the mean.

FIG. 12B is a bar graph showing caspase3+ and viability percentages (%) of total Lin− cells following (shaded bars) and not following (unshaded bars) treatment with 6-acetyl-8-cyclopentyl-5-methyl-2-(5-piperazin-1-yl-pyridin-2-ylamino)-8H-pyrido-[2,3-d]pyrimidin-7-one (PD 0332991) for 48 hours and 5-bromo-2-deoxyuridine (BrdU) pulse for 24 hours. Error bars show standard error of the mean.

FIG. 12C is a bar graph showing the caspase3+ and viability percentages (% s) of hematopoietic stem cells (HSC) following (shaded bars) or not following (unshaded bars) treatment with 6-acetyl-8-cyclopentyl-5-methyl-2-(5-piperazin-1-yl-pyridin-2-ylamino)-8H-pyrido-[2,3-d]pyrimidin-7-one (PD 0332991) for 48 hours and 5-bromo-2-deoxyuridine (BrdU) pulse for 24 hours. Error bars show standard error of the mean.

FIG. 12D is a bar graph showing the frequency of Lin− cells in myeloid, erythroid, and lymphoid progenitors after 48 hours of treatment with (shaded bars) or without (unshaded bars) 6-acetyl-8-cyclopentyl-5-methyl-2-(5-piperazin-1-yl-pyridin-2-ylamino)-8H-pyrido-[2,3-d]pyrimidin-7-one (PD 0332991). *p,0.05; **p<0.01, ***p<0.001. Error bars show standard error of the mean.

FIG. 12E is a bar graph showing the number of cobblestone area forming cells (CAFCs) at 1, 2, and 5 weeks after bone marrow harvest in untreated mice (unshaded bars; N=8) or mice treated with daily oral gavage with 6-acetyl-8-cyclopentyl-5-methyl-2-(5-piperazin-1-yl-pyridin-2-ylamino)-8H-pyrido-[2,3-d]pyrimidin-7-one (PD 0332991) for 2 days prior to bone marrow harvest (shaded bars; N=9). CAFCs are per 1×10⁵ bone marrow mononuclear cells (BM-MNCs). Error bars are +/− the standard error of the mean of pooled samples measured in duplicate.

FIG. 13A is a schematic diagram showing a treatment schedule of 6-acetyl-8-cyclopentyl-5-methyl-2-(5-piperazin-1-yl-pyridin-2-ylamino)-8H-pyrido-[2,3-d]pyrimidin-7-one (PD 0332991) in initial, multi-dose radio-protection experiments. Mice were dosed with PD 0332991 28 hours prior to (−28 hours), 4 hours prior to (−4 hours), and 20 hours following (+20 hours) ionizing radiation treatment.

FIG. 13B shows Kaplan Meier analysis of mice exposed to 7.5 gray (Gy) total body irradiation (TBI). The survival curve for mice treated with multiple doses of 6-acetyl-8-cyclopentyl-5-methyl-2-(5-piperazin-1-yl-pyridin-2-ylamino)-8H-pyrido-[2,3-d]pyrimidin-7-one (PD 0332991 as per FIG. 13A; 20 hours prior, 4 hours prior and 20 hours following TBI; N=9) is shown with the solid line. The survival curve for mice that had not been treated with PD 0332991 (N=9) is shown with the dotted line. P-value is determined with the log-rank test.

FIG. 13C shows Kaplan Meier analysis of mice exposed to 7.5 gray (Gy) total body irradiation (TBI) for mice treated with multiple doses of 6-acetyl-8-cyclopentyl-5-methyl-2-(5-piperazin-1-yl-pyridin-2-ylamino)-8H-pyrido-[2,3-d]pyrimidin-7-one (PD 0332991) at −28, −4, and +20 hours relative to the time of irradiation. The untreated animals (N=16) are shown by the dashed line, and the treated animals are shown by the solid line. Significance (***p<0.001) was determined by the log-rank test.

FIG. 13D shows Kaplan Meier analysis of mice exposed to 7.5 gray (Gy) total body irradiation (TBI) for mice treated with a single dose of 6-acetyl-8-cyclopentyl-5-methyl-2-(5-piperazin-1-yl-pyridin-2-ylamino)-8H-pyrido-[2,3-d]pyrimidin-7-one (PD 0332991) at the same time (0 hours) as irradiation. The untreated animals (N=16) are shown by the dashed line, and the treated animals (N=8) are shown by the solid line. Significance (**p<0.01) was determined by the log-rank test.

FIG. 13E shows Kaplan Meier analysis of mice exposed to 7.5 gray (Gy) total body irradiation (TBI) for mice treated with a single dose of 6-acetyl-8-cyclopentyl-5-methyl-2-(5-piperazin-1-yl-pyridin-2-ylamino)-8H-pyrido-[2,3-d]pyrimidin-7-one (PD 0332991) four hours prior (−4 hours) to irradiation. The untreated animals (N=16) are shown by the dashed line, and the treated animals (N=9) are shown by the solid line. Significance (**p<0.01) was determined by the log-rank test.

FIG. 13F shows Kaplan Meier analysis of mice exposed to 7.5 gray (Gy) total body irradiation (TBI) for mice treated with a single dose of 6-acetyl-8-cyclopentyl-5-methyl-2-(5-piperazin-1-yl-pyridin-2-ylamino)-8H-pyrido-[2,3-d]pyrimidin-7-one (PD 0332991) twenty hours (+20 hours) after irradiation. The untreated animals (N=16) are shown by the dashed line, and the treated animals (N=10) are shown by the solid line. Significance (*p<0.05) was determined by the log-rank test.

FIG. 13G is a bar graph showing the hematocrit or cell counts of different lineages of blood cells from mice 21 days following exposure to a lethal dose (7.5 Gy) of ionizing radiation (IR). Data for mice treated with 6-acetyl-8-cyclopentyl-5-methyl-2-(5-piperazin-1-yl-pyridin-2-ylamino)-8H-pyrido-[2,3-d]pyrimidin-7-one (PD 0332991) is shown in the darkly shaded bars, while data for irradiated mice that had not been treated with PD 0332991 is shown in the unshaded bars. For comparison, data from mice that had not been exposed to IR is also shown (lightly shaded bars). The data indicates that treatment with PD 0332991 protects all lineages of blood cells. Myeloid cell count is the sum of granulocytes and monocytes. *p,0.05; **p<0.01, ***p<0.001. # indicates that the maximum value of the cohort is shown in lieu of error bars where cells numbers were too few to reliably quantify. Error bars show standard error of the mean.

FIG. 14A shows Kaplan Meier analysis of inbred C3H mice exposed to 7.5 gray (Gy) total body irradiation (TBI). The survival curve for mice treated with a single dose of 6-acetyl-8-cyclopentyl-5-methyl-2-(5-piperazin-1-yl-pyridin-2-ylamino)-8H-pyrido-[2,3-d]pyrimidin-7-one (PD 0332991) four hours prior to TBI is shown with the solid line (N=9). The survival curve for mice that had not been treated with PD 0332991 is shown with the dotted line (N=9). P-values determined by the log-rank test.

FIG. 14B shows Kaplan Meier analysis of inbred C57BI/6 mice exposed to 6.5 gray (Gy) total body irradiation (TBI). The survival curve for mice treated with a single dose of 6-acetyl-8-cyclopentyl-5-methyl-2-(5-piperazin-1-yl-pyridin-2-ylamino)-8H-pyrido-[2,3-d]pyrimidin-7-one (PD 0332991) four hours prior to TBI is shown with the solid line. The survival curve for mice that had not been treated with PD 0332991 is shown with the dotted line.

FIG. 14C shows Kaplan Meier analysis of mice exposed to 8.5 gray (Gy) total body irradiation (TBI). The survival curves are shown for mice treated with a single dose of 6-acetyl-8-cyclopentyl-5-methyl-2-(5-piperazin-1-yl-pyridin-2-ylamino)-8H-pyrido-[2,3-d]pyrimidin-7-one (PD 0332991) 4 hours prior to TBI (N=17, solid line) and for mice that had been treated with TBI but not treated with PD 0332991 (N=13, dotted line). P-values determined by the log-rank test.

FIG. 15 is a set of graphs showing hematocrit or cell counts of different lineages of blood cells from mice treated (shaded circles) or untreated (unshaded squares) with 6-acetyl-8-cyclopentyl-5-methyl-2-(5-piperazin-1-yl-pyridin-2-ylamino)-8H-pyrido-[2,3-d]pyrimidin-7-one (PD0332991) and exposed to a sub-lethal dose (6.5 gray (Gy)) of total body irradiation (TBI). Data was prepared from weekly complete blood counts on tail vein bleeds. Asterisk(s) indicate statistical significance determined by a 2-sided t-test. Error bars are +/− the standard error of the mean.

FIG. 16 is set of bar graphs showing the hematocrit or cell counts of different lineages of blood cells from mice 143-242 days following exposure to a lethal dose (7.5 gray (Gy)) or sub-lethal dose (6.5 Gy) of total body irradiation (TBI). Data for mice treated with 6-acetyl-8-cyclopentyl-5-methyl-2-(5-piperazin-1-yl-pyridin-2-ylamino)-8H-pyrido-[2,3-d]pyrimidin-7-one (PD 0332991) prior to 6.5 Gy TBI is shown in the solid bars and data for mice treated with PD 0332991 prior to 7.5 Gy TBI is shown in the stippled bars. For comparison, data for mice that had been exposed to 6.5 Gy TBI, but that had not been treated with PD 0332991 is shown in the striped bars. Data for mice that had not been treated with PD 0332991 or TBI is shown in the unshaded bars. Myeloid cell count is the sum of granulocytes and monocytes.

FIG. 17 is a series of graphs showing complete blood count (CBC) data from mice treated by daily oral gavage with 150 mg/kg of 6-acetyl-8-cyclopentyl-5-methyl-2-(5-piperazin-1-yl-pyridin-2-ylamino)-8H-pyrido-[2,3-d]pyrimidin-7-one (PD0332991) for 12 days. Data are shown as a moving average with each point representing the mean of three consecutive CDCs. Error bars are +/− of the standard error of the mean for all data points associated with the moving average. The solid black bar in the lower left of each graph indicates the duration of the PD0332991 treatment. Data from PD0332991 treated mice is shown by the unshaded squares. For comparison, data from mice that had not been treated with PD0332991 is shown in shaded circles.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a compound” or “a cell” includes a plurality of such compounds or cells, and so forth.

The term “and/or” when used in describing two items or conditions, e.g., CDK4 and/or CDK6, refers to situations where both items or conditions are present or applicable and to situations wherein only one of the items or conditions is present or applicable. Thus, a CDK4 and/or CDK6 inhibitor can be a compound that inhibits both CDK4 and CDK6, a compound that inhibits only CDK4, or a compound that only inhibits CDK6.

By “healthy cell” or “normal cell” is meant any cell in a subject that does not display the symptoms or markers of a disease. In some embodiments, the healthy cell is a hematopoietic stem or progenitor cell. Progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs).

As used herein the term “ionizing radiation” refers to radiation of sufficient energy that, when absorbed by cells and tissues, typically induces formation of reactive oxygen species and DNA damage. Ionizing radiation can include X-rays, gamma rays, and particle bombardment (e.g., neutron beam, electron beam, protons, mesons, and others), and is used for purposes including, but not limited to, medical testing and treatment, scientific purposes, industrial testing, manufacturing and sterilization, and weapons and weapons development. Radiation is generally measured in units of absorbed dose, such as the rad or gray (Gy), or in units of dose equivalence, such as rem or sievert (Sv).

By “at risk of incurring exposure to ionizing radiation” is meant a subject scheduled for (such as by scheduled radiotherapy sessions) exposure to IR in the future or a subject having a chance of being exposed to IR inadvertently in the future. Inadvertent exposure includes accidental or unplanned environmental or occupational exposure (e.g., terrorist attack with a radiological weapon or exposure to a radiological weapon on the battlefield).

By “effective amount of an inhibitor compound” is meant an amount effective to reduce or eliminate the toxicity associated with radiation in healthy hematopoietic stem/progenitor cells in the subject. In some embodiments, the effective amount is the amount required to temporarily (e.g., for a few hours or days) inhibit the proliferation of hematopoietic stem cells (i.e., to induce a quiescent state in hematopoietic stem cells) in the subject.

By “long-term hematological toxicity” is meant hematological toxicity affecting a subject for a period lasting more than one or more weeks, months or years following administration of the selective CDK4/6 inhibitor. Long-term hematological toxicity can result in bone marrow disorders that can cause the ineffective production of blood cells (i.e., myelodysplasia) and/or lymphocytes (i.e., lymphopenia, the reduction in the number of circulating lymphocytes, such as B- and T-cells). Hematological toxicity can be observed, for example, as anemia, reduction in platelet count (i.e., thrombocytopenia) or reduction in white blood cell count (i.e., neutropenia). In some cases, myelodysplasia can result in the development of leukemia. Long-term toxicity related to ionizing radiation can also damage other self renewing cells in a subject, in addition to hematological cells. Thus, long-term toxicity can also lead to graying and frailty.

By “free of” is meant that subjects treated with a selective CDK4/6 inhibitor by the presently disclosed methods do not display any detectable signs or symptoms of long-term hematologic toxicity or display signs or symptoms of long-term hematologic toxicity that are significantly reduced (e.g., reduced 10 times, or reduced 100 times or more) compared to the signs/symptoms that would be displayed by subjects treated with IR who did not receive a dose or doses of a CDK4/6 inhibitor.

“Free of” can also refer to a selective CDK4/6 inhibitor compound not having an undesired or off-target effect, particularly when used in vivo or assessed via a cell-based assay. Thus, “free of” can refer to a selective CDK4/6 inhibitor not having off-target effects such as, but not limited to, long term toxicity, anti-oxidant effects, estrogenic effects, tyrosine kinase inhibitory effects, inhibitory effects on CDKs other than CDK4/6, and cell cycle arrest in CDK4/6-independent cells.

A CDK4/6 inhibitor that is “substantially free” of off-target effects is a CDK4/6 inhibitor that can have some minor off-target effects that do not interfere with the inhibitor's ability to provide protection from cytotoxic compounds in CDK4/6-dependent cells. For example, a CDK4/6 inhibitor that is “substantially free” of off-target effects can have some minor inhibitory effects on other CDKs (e.g., IC₅₀s for CDK1 or CDK2 that are >0.5 μM; >1.0 μM, or >5.0 μM), so long as the inhibitor provides selective G1 arrest in CDK4/6-dependent cells.

By “reduced” and “prevented” or grammatical variations thereof mean, respectively, lessening the undesirable side effects of a medical treatment or keeping the undesirable side effects from occurring completely.

In some embodiments, the subject treated in the presently disclosed subject matter is desirably a human subject, although it is to be understood the methods described herein are effective with respect to all vertebrate species, which are intended to be included in the term “subject.”

More particularly, provided herein is the treatment of mammals, such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Thus, embodiments of the methods described herein include the treatment of livestock, including, but not limited to, domesticated swine (pigs and hogs), ruminants, horses, poultry, and the like.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

“Aryloxyl” or “aryloxy” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” or “aralkyloxy” refers to an aralkyl-O— group wherein the aralkyl group is as previously described. An exemplary aralkyloxyl group is benzyloxyl.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some embodiments, the amino group is —NH₂. “Aminoalkyl” and “aminoaryl” refer to —NR′R″ groups wherein R′ is defined as for the amino group and R″ is substituted or unsubstituted alkyl or aryl, respectively.

“Acylamino” refers to an acyl-NH— group wherein acyl is as previously described.

The term “carbonyl” refers to the —(C═O)— or a double bonded oxygen substituent attached to a carbon atom of a previously named parent group.

The term “carboxyl” refers to the —COOH group.

The terms “halo”, “halide”, or “halogen” as used herein refer to fluoro, chloro, bromo, and iodo groups.

The terms “hydroxyl” and “hydroxy” refer to the —OH group.

The term “oxo” refers to a compound described previously herein wherein a carbon atom is replaced by an oxygen atom.

The term “cyano” refers to the —CN group.

The term “nitro” refers to the —NO₂ group.

The term “thio” refers to a compound described previously herein wherein a carbon or oxygen atom is replaced by a sulfur atom.

### II. Hematopoietic Stem Cells and Cyclin-Dependent Kinase Inhibitors

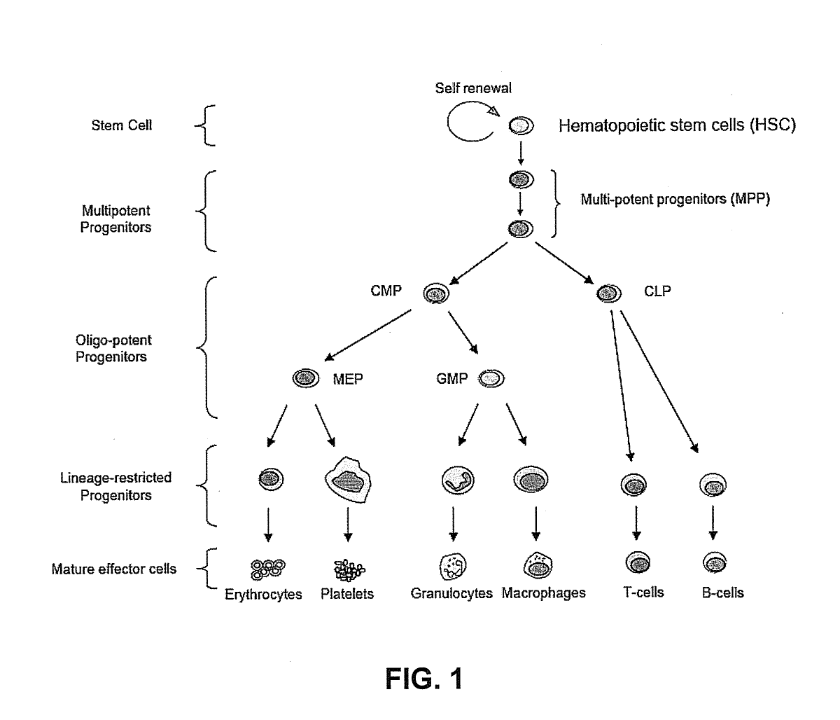

Tissue-specific stem cells are capable of self-renewal, meaning that they are capable of replacing themselves throughout the adult mammalian lifespan through regulated replication. Additionally, stem cells divide asymmetrically to produce “progeny” or “progenitor” cells that in turn produce various components of a given organ. For example, in the hematopoietic system, the hematopoietic stem cells give rise to progenitor cells which in turn give rise to all the differentiated components of blood (e.g., white blood cells, red blood cells, and platelets). See FIG. 1.

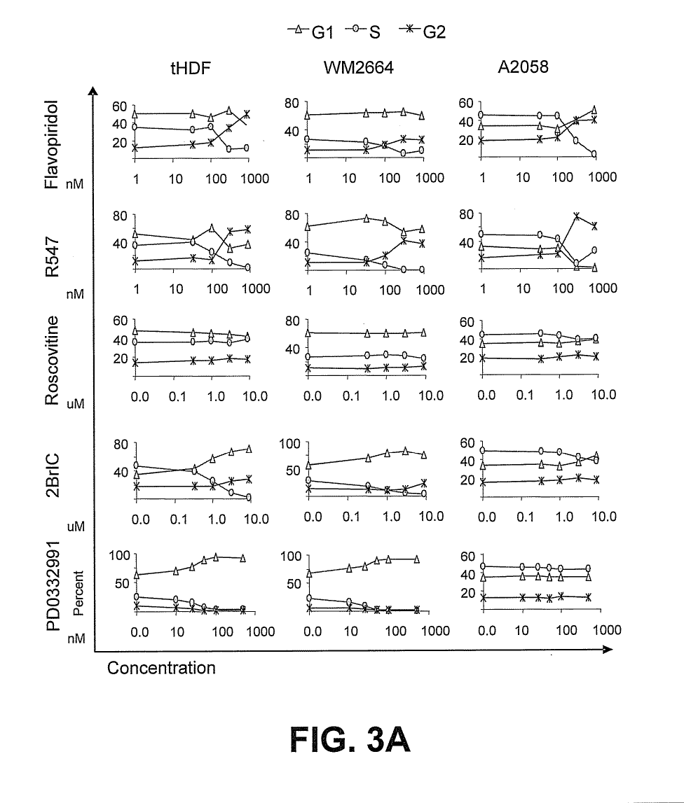

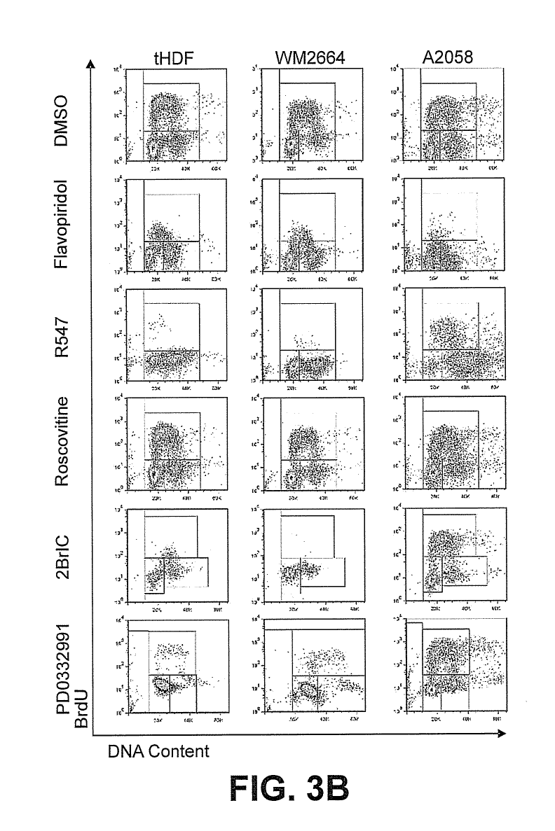

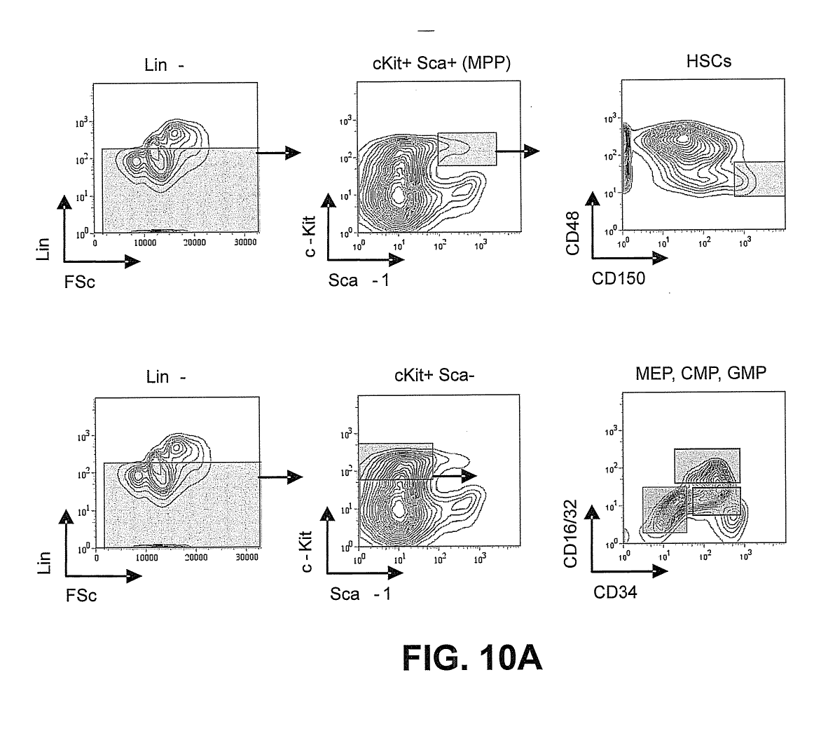

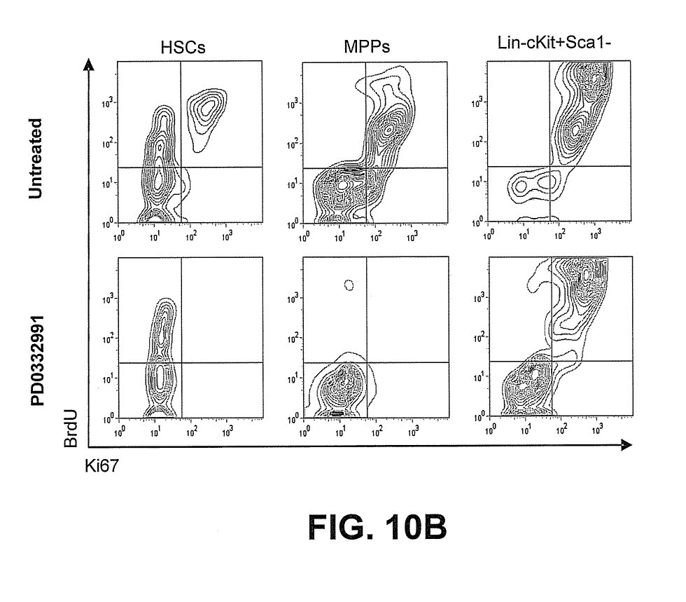

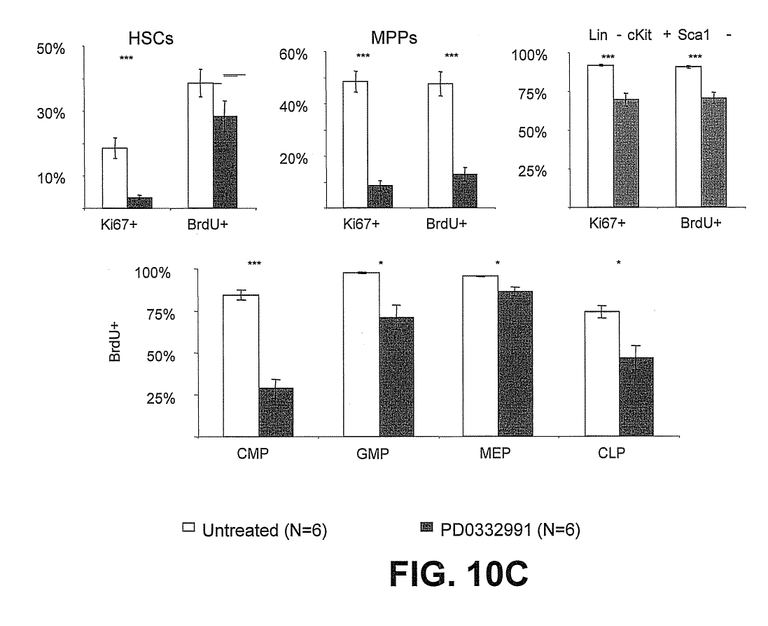

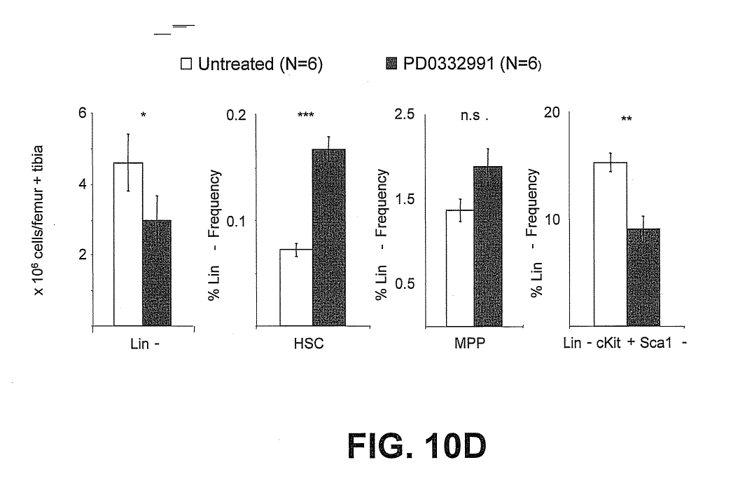

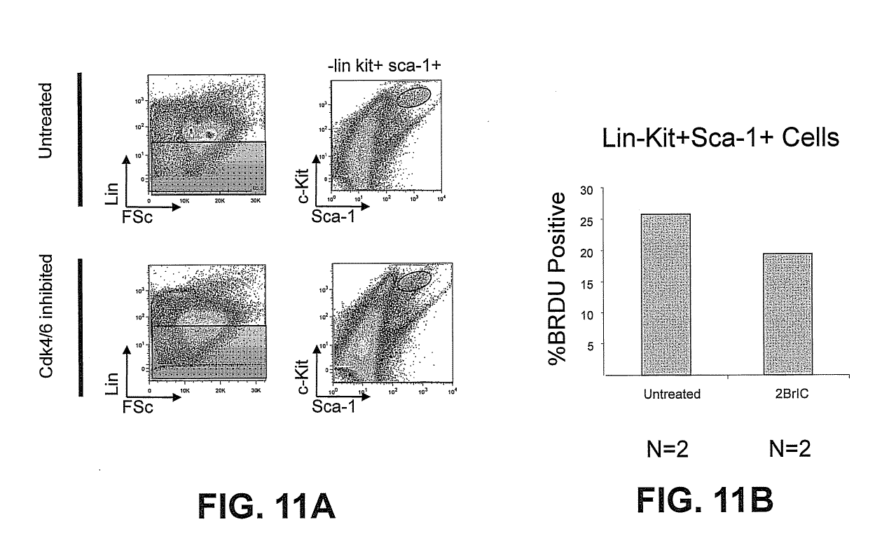

The presently disclosed subject matter relates to the specific biochemical requirements of early hematopoietic stem/progenitor cells (HSPC) in the adult mammal. In particular, it has been found that these cells require the enzymatic activity of the proliferative kinases cyclin-dependent kinase 4 (CDK4) and/or cyclin-dependent kinase 6 (CDK6) for cellular replication. In contrast, the vast majority of proliferating cells in adult mammals (e.g., the more differentiated blood-forming cells in the bone marrow) do not require the activity of CDK4 and/or CDK6 (i.e., CDK4/6). These differentiated cells can proliferate in the absence of CDK4/6 activity by using other proliferative kinases, such as cyclin-dependent kinase 2 (CDK2) or cyclin-dependent kinase 1 (CDK1). Therefore, treatment of adult mice with a specific CDK4/6 inhibitor leads to inhibition of proliferation (i.e., pharmacologic quiescence (PQ)) in very restricted stem and progenitor compartments. For instance, transient treatment with PD 0332991, a selective CDK4/6 inhibitor, renders hematopoietic stem cells and their associated hematopoietic progenitor cells quiescent. See FIGS. 10B-10C and 11A-11B. In particular, treatment leads to selective G1 arrest in CDK4/6-dependent cells. See FIG. 3A.

The presently disclosed subject matter relates to methods of protecting healthy cells (e.g., in a subject) from the toxicity of ionizing radiation by the administration of selective CDK4/6 inhibitors. Without being bound to any one theory, administration of such inhibitors is expected to force stem cells in the subject into PQ. Cells that are quiescent are more resistant to the DNA damaging effect of radiation than proliferating cells. As the most acute and severe toxicities of ionizing radiation are through its effect on stem and progenitor cells, making the stem and progenitor cells radio-resistant can protect the entire organism from the acute and chronic toxicities of radio-therapy.

Thus, in some embodiments, the presently disclosed subject matter provides methods for protection of mammals from the acute and chronic toxic effects of ionizing radiation by forcing hematopoietic stem and progenitor cells (HSPCs) into a quiescent state by transient (e.g., over a less than about 48, 36, 24, 20, 16, 12, 10, 8, 6, 4, 2, or 1 hour period) treatment with a non-toxic, selective CDK4/6 inhibitor (e.g., orally available, selective CDK4/6 inhibitors). See FIGS. 10A-10D, 11A, 11B, and 12A-12E. HSPCs recover from this period of transient quiescence, and then function normally after treatment with the inhibitor is stopped. During the period of quiescence, the stem and progenitor cells are protected from the effects of ionizing radiation. The ability to protect stem/progenitor cells is desirable both in the treatment of cancer (where patients are given high doses of ionizing radiation) and in radiation mitigation (where individuals are exposed to large doses of radiation in an industrial accident or after explosion of a nuclear device).

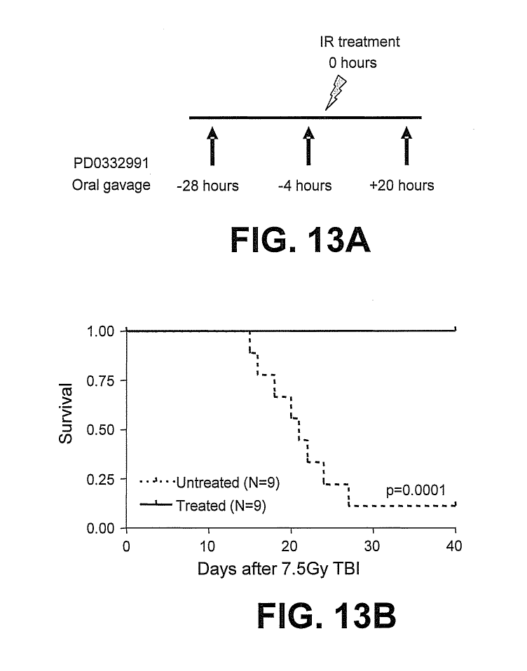

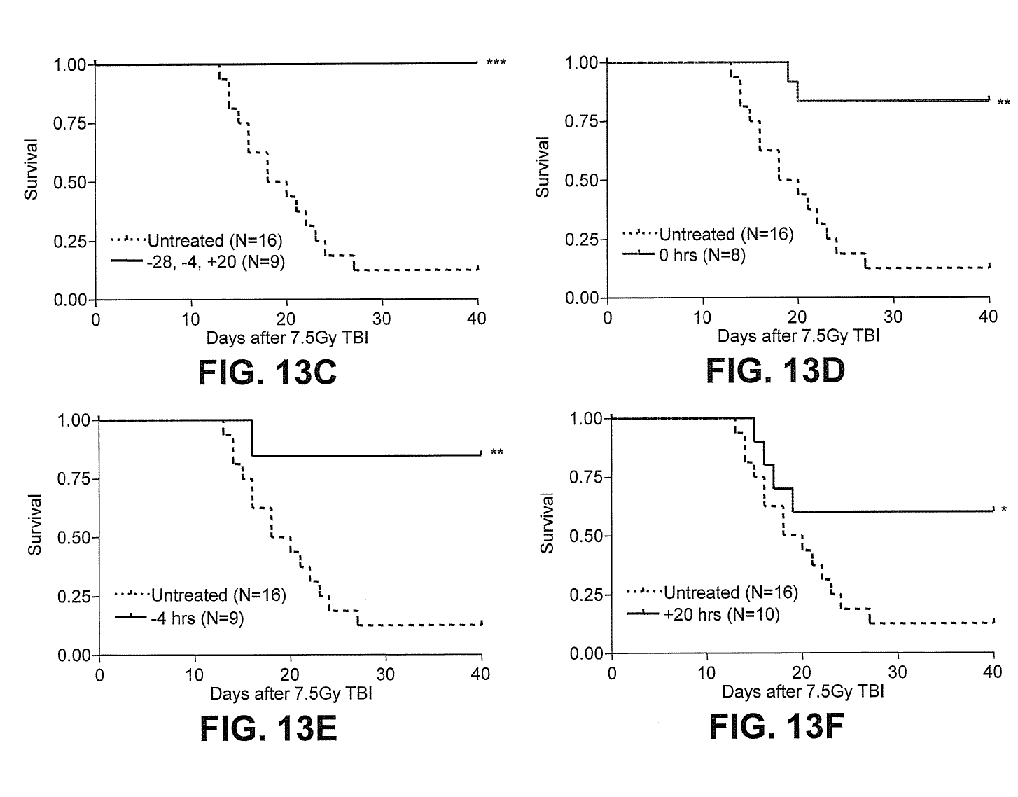

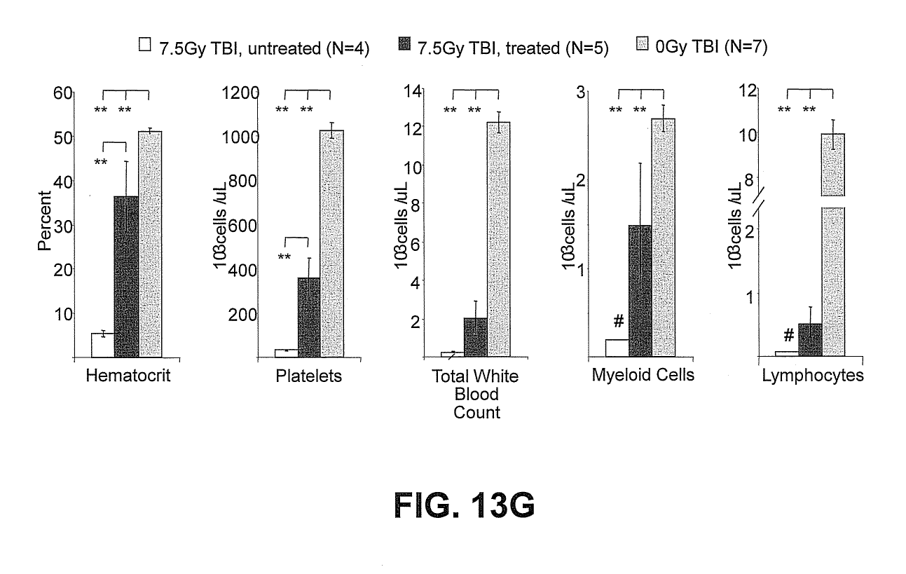

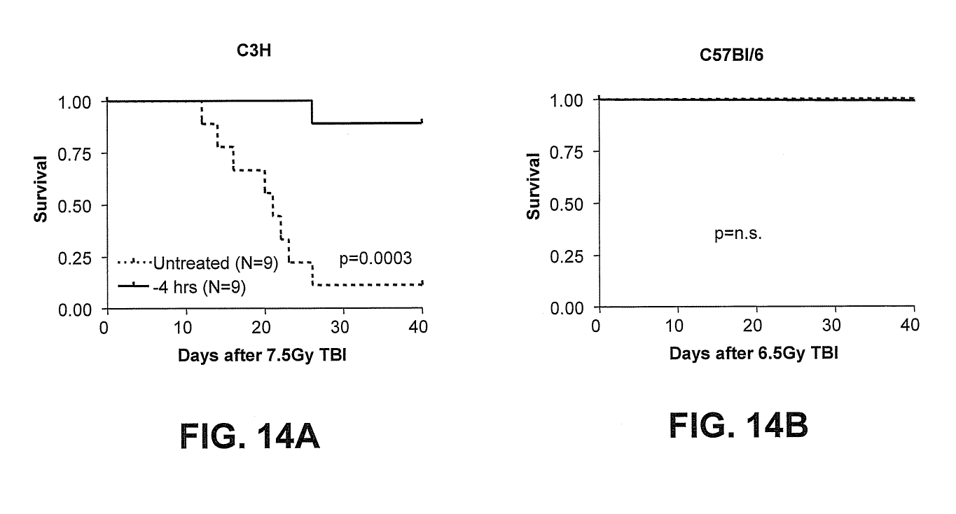

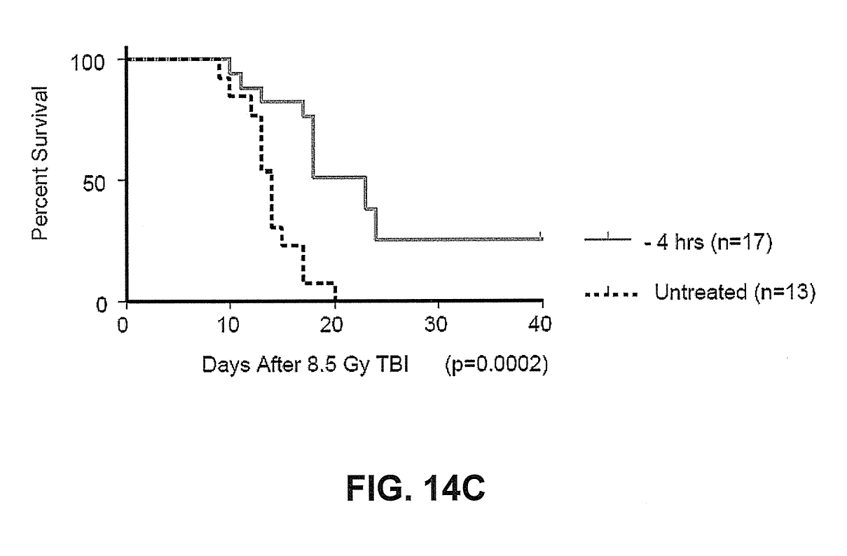

By way of example and not limitation, the presently disclosed subject matter relates to the finding that transient treatment (treatment for <48, 36, 24, 20, 16, 12, 10, 8, 6, 4, 2, or 1 hours) of mice with as little as a single oral dose of PD 0332991 at a time point close to the time of exposure to ionizing radiation affords marked radio-protection. See FIGS. 13A-13F, 14A, and 14C. In initial studies, as illustrated in FIGS. 13A-13C, mice were treated with multiple doses of PD 0332991 (at 20 hours and 4 hours prior to exposure to IR as well as at 20 hours post-IR). When untreated mice were exposed to 7.5 Gy of IR, more than 90% die within 40 days from bone marrow failure (e.g., neutropenia and anemia). In contrast, nearly 100% of the treated mice survive the dose. Enhanced survival was also seen when PD 0332991 was given as a singe dose four hours prior to IR. See FIG. 13E. At even higher doses of IR (8.5 Gy), all untreated mice succumb whereas about 30% of mice treated with PD 0332991 survive, and even the treated mice that die do so at a later date following IR. See FIG. 14C. Such a delay of lethal toxicity could be clinically significant in humans exposed to radiation, as it can allow time for other supportive medical treatments, including, but not limited to stem cell transplants.

According to the presently disclosed subject matter, radiation protection with selective CDK4/6 inhibitors can be achieved by a number of different dosing schedules. In addition to multi-dosing schedules or single pretreatment, concomitant treatment can also be effective. See FIGS. 13B-13F. Further, treatment even after exposure to ionizing radiation can afford radio-protection. See FIG. 13F. Thus, the dosing schedule can be flexible. Of particular importance with regard to accidental or unanticipated exposure to IR, especially in cases of exposure involving large numbers of subjects, dosing with selective CDK4/6 inhibitors, such as a pyrido[2,3-d]pyrimidin-7-one (e.g., PD0332991), can be performed more than 20 hours following radiation exposure.

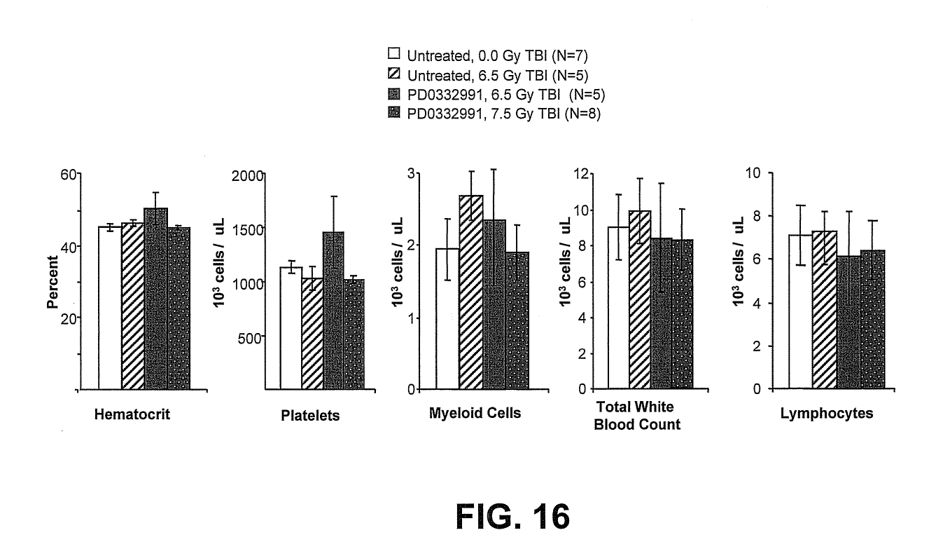

Radio-protection with selective CDK4/6 inhibitors, as shown by the exemplary compounds 2BrIC and PD 0332991, is associated with marked bone marrow protection, which in turn leads to a more rapid recovery of peripheral blood cell counts (hematocrit, platelets, lymphocytes, and myeloid cells) after IR. See FIG. 13G. This effect is comparable to that seen with the use of exogenous growth factors (e.g., granulocyte colony-stimulating factor (GCSF) and erythropoietin), although treatment with selective CDK4/6 inhibitor compounds has some advantages in that it ameliorates suppression of platelet count, which no previously reported treatment is capable of doing effectively. Treatment with selective CDK4/6 inhibitors also protects stem cells and their progenitors from damage rather than forcing them to proliferate at a faster rate. This is important because enforced proliferation can increase late and long-term bone marrow toxicities seen in humans and mice after growth factor support intended to ameliorate the effects of DNA damage. See Herodin et al., Blood, 2003, 101, 2609-2616; Hershman et al., J. Natl. Cancer Inst., 2007, 99, 196-205; and Le Deley et al., J. Clin. Oncol., 2007, 25, 292-300. No late toxicity is seen in mice treated with PD 0332991 at the time of a sub-lethal dose (6.5 Gy) of TBI even when examined more than 100-200 days after IR exposure. See FIG. 16.

Several advantages can result from radio-protective methods involving selective CDK4/6 inhibitors. The reduction in radio-toxicity afforded by the selective CDK4/6 inhibitors can allow for dose intensification (e.g., more therapy can be given in a fixed period of time) in medically related IR therapies, which will translate to better efficacy. Therefore, the presently disclosed methods can result in radio-therapy regimens that are less toxic and more effective. Also, in contrast to protective treatments with exogenous biological growth factors, selective CDK4/6 inhibitors include many orally available small molecules, which can be formulated for administration via a number of different routes. When appropriate, such small molecules can be formulated for oral, topical, intranasal, inhalation, intravenous or any other form of administration. Further, as opposed to biologics, stable small molecules can be more easily stockpiled and stored. Thus, the selective CDK4/6 inhibitor compounds can be more easily and cheaply kept on hand in emergency rooms where subjects of IR exposure can report or at sites where radiation exposure is particularly likely to occur: at nuclear power plants, on nuclear powered vessels, at military installations, near battlefields, etc.

As used herein the term “selective CDK4/6 inhibitor compound” refers to a compound that selectively inhibits at least one of CDK4 and CDK6, or whose predominant mode of action is through inhibition of CDK4 and/or CDK6. Thus, selective CDK4/6 inhibitors are compounds that generally have a lower 50% inhibitor concentration (IC₅₀) for CDK4 and/or CDK6 than for other kinases. In some embodiments, the selective CDK4/6 inhibitor can have an IC₅₀ for CDK4 or CDK6 that is at least 2, 3, 4, 5, 6, 7, 8, 9, or 10 times lower than the compound's IC₅₀s for other CDKs (e.g., CDK1 and CDK2). In some embodiments, the selective CDK4/6 inhibitor can have an IC₅₀ for CDK4 or CDK6 that is at least 20, 30, 40, 50, 60, 70, 80, 90, or 100 times lower than the compound's IC₅₀s for other CDKs. In some embodiments; the selective CDK4/6 inhibitor can have an IC₅₀ that is more than 100 times or more than 1000 times less than the compound's IC₅₀s for other CDKs.

In some embodiments, the selective CDK4/6 inhibitor is a compound that can induce selective G1 arrest in CDK4/6-dependent cells (e.g., as measured in a cell-based in vitro assay). Thus, when treated with the selective CDK4/6 inhibitor compound according to the presently disclosed methods, the percentage of CDK4/6-dependent cells in the G1 phase increase, while the percentage of CDK4/6-dependent cells in the G2/M phase and S phase decrease. In some embodiments, the selective CDK4/6 inhibitor is a compound that induces substantially pure (i.e., “clean”) G1 cell cycle arrest in the CDK4/6-dependent cells (e.g., wherein treatment with the selective CDK4/6 inhibitor induces cell cycle arrest such that the majority of cells are arrested in G1 as defined by standard methods (e.g. propidium iodide (PI) staining or others) with the population of cells in the G2/M and S phases combined is 20%, 15%, 12%, 10%, 8%, 6%, 5%, 4%, 3%, 2%, 1% or less of the total cell population). Methods of assessing the cell phase of a population of cells are known in the art (see, for example, in U.S. Patent Application Publication No. 2002/0224522) and include cytometric analysis, microscopic analysis, gradient centrifugation, elutriation, fluorescence techniques including immunofluorescence, and combinations thereof. Cytometric techniques include exposing the cell to a labelling agent or stain, such as DNA-binding dyes, e.g., PI, and analyzing cellular DNA content by flow cytometry. Immunofluorescence techniques include detection of specific cell cycle indicators such as, for example, thymidine analogs (e.g., 5-bromo-2-deoxyuridine (BrdU) or an iododeoxyuridine), with fluorescent antibodies.

While staurosporine, a non-specific kinase inhibitor, has been reported to indirectly induce G1 arrest in some cell types (see Chen et al., J. Nat. Cancer Inst., 92, 1999-2008 (2000)), the presently disclosed use of selective CDK4/6 inhibitors to directly and selectively induce G1 cell cycle arrest in cells, such as specific fractions of HSPCs, can provide chemoprotection with reduced long term toxicity and without the need for prolonged (e.g., 48 hour or longer) treatment with the inhibitor prior to exposure with the DNA damaging compound. In particular, while some nonselective kinase inhibitors can cause G1 arrest in some cell types by decreasing CDK4 protein levels, benefits of the presently disclosed methods are, without being bound to any one theory, believed to be due at least in part to the ability of selective CDK4/6 inhibitors to directly inhibit the kinase activity of CDK4/6 in HSPCs without decreasing their cellular concentration.

In some embodiments, the selective CDK4/6 inhibitor compound is a compound that is substantially free of off-target effects, particularly related to inhibition of kinases other than CDK4 and or CDK6. In some embodiments, the selective CDK4/6 inhibitor compound is a poor inhibitor (e.g., >1 μM IC₅₀) of CDKs other than CDK4/6 (e.g., CDK1 and CDK2). In some embodiments, the selective CDK4/6 inhibitor compound does not induce cell cycle arrest in CDK4/6-independent cells. In some embodiments, the selective CDK4/6 inhibitor compound is a poor inhibitor (e.g., >1 μM IC₅₀) of tyrosine kinases. Additional, undesirable off-target effects include, but are not limited to, long term toxicity, anti-oxidant effects, and estrogenic effects.

Anti-oxidant effects can be determined by standard assays known in the art. For example, a compound with no significant anti-oxidant effects is a compound that does not significantly scavenge free-radicals, such as oxygen radicals. The anti-oxidant effects of a compound can be compared to a compound with known anti-oxidant activity, such as genistein. Thus, a compound with no significant anti-oxidant activity can be one that has less than about 2, 3, 5, 10, 30, or 100 fold anti-oxidant activity relative to genistein. Estrogenic activities can also be determined via known assays. For instance, a non estrogenic compound is one that does not significantly bind and activate the estrogen receptor. A compound that is substantially free of estrogenic effects can be one that has less than about 2, 3, 5, 10, 20, or 100 fold estrogenic activity relative to a compound with estrogenic activity, e.g., genistein.

Selective CDK4/6 inhibitors that can be used according to the presently disclosed methods include any known small molecule (e.g., <1000 Daltons, <750 Daltons, or less than <500 Daltons), selective CDK4/6 inhibitor, or pharmaceutically acceptable salt thereof. In some embodiments, the selective CDK4/6 inhibitor is a non-naturally occurring molecule (i.e., a molecule not found or existing in nature). In some embodiments, the inhibitor is not staurosporine or genistein. A number of different chemical classes of compounds have been reported in the literature as having CDK4/6 inhibitory ability (e.g., in non-cell based in vitro assays). Thus, it is believed that selective CDK4/6 inhibitors useful in the presently disclosed methods can include, but are not limited to, pyrido[2,3-d]pyrimidines (e.g., pyrido[2,3-d]pyrimidin-7-ones and 2-amino-6-cyano-pyrido[2,3-d]pyrimidin-4-ones), triaminopyrimidines, aryl[a]pyrrolo[3,4-d]carbazoles, nitrogen-containing heteroaryl-substituted ureas, 5-pyrimidinyl-2-aminothiazoles, benzothiadiazines, acridinethiones, and isoquinolones.

In some embodiments, the pyrido[2,3-d]pyrimidine is a pyrido[2,3-d]pyrimidinone. In some embodiments the pyrido[2,3-d]pyrimidinone is pyrido[2,3-d]pyrimidin-7-one. In some embodiments, the pyrido[2,3-d]pyrimidin-7-one is substituted by an aminoaryl or aminoheteroaryl group. In some embodiments, the pyrido[2,3-d]pyrimidin-7-one is substituted by an aminopyridine group. In some embodiments, the pyrido[2,3-d]pyrimidin-7-one is a 2-(2-pyridinyl)amino pyrido[2,3-d]pyrimidin-7-one. For example, the pyrido[2,3-d]pyrimidin-7-one compound can have a structure of Formula (II) as described in U.S. Patent Publication No. 2007/0179118 to Barvian et al., herein incorporated by reference in its entirety. In some embodiments, the pyrido[2,3-d]pyrimidine compound is 6-acetyl-8-cyclopentyl-5-methyl-2-(5-piperazin-1-yl-pyridin-2-ylamino)-8H-pyrido-[2,3-d]pyrimidin-7-one (i.e., PD 0332991) or a pharmaceutically acceptable salt thereof. See Toogood et al., J. Med. Chem., 2005, 48, 2388-2406.

In some embodiments, the pyrido[2,3-d]pyrimidinone is a 2-amino-6-cyano-pyrido[2,3-d]pyrimidin-4-ones. Selective CDK4/6 inhibitors comprising a 2-amino-6-cyano-pyrido[2,3-d]pyrimidin-4-one are described, for example, by Tu et al. See Tu et al., Bioorg. Med. Chem. Lett., 2006, 16, 3578-3581.

As used herein, “triaminopyrimidines” are pyrimidine compounds wherein at least three carbons in the pyrimidine ring are substituted by groups having the formula —NR₁R₂, wherein R₁ and R₂ are independently selected from the group consisting of H, alkyl, aralkyl, cycloalkyl, heterocycle, aryl, and heteroaryl. Each R₁ and R₂ alkyl, aralkyl, cycloalkyl, aryl and heteroaryl groups can be further substituted by one or more hydroxyl, halo, amino, alkyl, aralkyl, cycloalkyl, heterocyclic, aryl or heteroaryl groups. In some embodiments, at least one of the amino groups is an alkylamino group having the structure —NHR, wherein R is C₁-C₆ alkyl. In some embodiments, at least one amino group is a cycloalkylamino group or a hydroxyl-substituted cycloalkylamino group having the formula —NHR wherein R is C₃-C₇ cycloalkyl, substituted or unsubstituted by a hydroxyl group. In some embodiments, at least one amino group is a heteroaryl-substituted amino group, wherein the heteroaryl group can be further substituted with an aryl group substituent.

Aryl[a]pyrrolo[3,4-d]carbazoles include, but are not limited to napthyl[a]pyrrolo[3,4-c]carbazoles, indolo[a]pyrrolo[3,4-c]carbazoles, quinolinyl[a]pyrrolo[3,4-c]carbazoles, and isoquinolinyl[a]pyrrolo[3,4-c]carbazoles. See e.g., Engler et al., Bioorg. Med. Chem. Lett., 2003, 13, 2261-2267; Sanchez-Martinez et al., Bioorg. Med. Chem. Lett., 2003, 13, 3835-3839; Sanchez-Martinez et al., Bioorg. Med. Chem. Lett., 2003, 13, 3841-3846; Zhu et al., Bioorg. Med. Chem. Lett., 2003, 13, 1231-1235; and Zhu et al., J. Med Chem., 2003, 46, 2027-2030. See also, U.S. Patent Publication Nos. 2003/0229026 and 2004/0048915. In some embodiments, the aryl[a]pyrrolo[3,4-d]carbazole is 2-bromo-12, 13-dihydro-5H-indolo[2,3-a]pyrrolo[3,4]-carbazole-5,6-dione (2BrIC).

Nitrogen-containing heteroaryl-substituted ureas are compounds comprising a urea moiety wherein one of the urea nitrogen atoms is substituted by a nitrogen-containing heteraryl group. Nitrogen-containing heteroaryl groups include, but are not limited to, five to ten membered aryl groups including at least one nitrogen atom. Thus, nitrogen-containing heteroaryl groups include, for example, pyridine, pyrrole, indole, carbazole, imidazole, thiazole, isoxazole, pyrazole, isothiazole, pyrazine, triazole, tetrazole, pyrimidine, pyridazine, purine, quinoline, isoquinoline, quinoxaline, cinnoline, quinazoline, benzimidazole, phthalimide and the like. In some embodiments, the nitrogen-containing heteroaryl group can be substituted by one or more alkyl, cycloalkl, heterocyclic, aralkyl, aryl, heteroaryl, hydroxyl, halo, carbonyl, carboxyl, nitro, cyano, alkoxyl, or amino group. In some embodiments, the nitrogen-containing heteroaryl substituted urea is a pyrazole-3-yl urea. The pyrazole can be further substituted by a cycloalkyl or heterocyclic group. In some embodiments, the pyrazol-3-yl urea is:

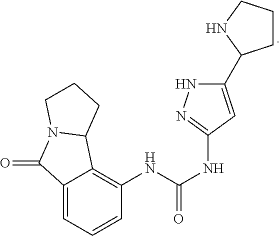

See Ikuta, et al., J. Biol. Chem., 2001, 276, 27548-27554. Additional ureas that can be used according to the presently disclosed subject matter include the biaryl urea compounds of Formula (I) described in U.S. Patent Publication No. 2007/0027147. See also, Honma et al., J. Med. Chem., 2001, 44, 4615-4627; and Honma et al., J. Med. Chem., 2001, 44, 4628-4640.

Suitable 5-pyrimidinyl-2-aminothiazole CDK4/6 inhibitors are described by Shimamura et al. See Shimamura et al., Bioorg. Med. Chem. Lett., 2006, 16, 3751-3754. In some embodiments, the 5-pyrimidinyl-2-aminothiazole has the structure:

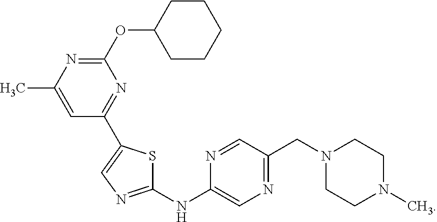

Useful benzothiadiazine and acridinethiones compounds include those, for example, disclosed by Kubo et al. See Kubo et al., Clin. Cancer Res. 1999, 5, 4279-4286 and in U.S. Patent Publication No. 2004/0006074, herein incorporated by reference in their entirety. In some embodiments, the benzothiadiazine is substituted by one or more halo, haloaryl, or alkyl group. In some embodiments, the benzothiadiazine is selected from the group consisting of 4-(4-fluorobenzylamino)-1,2,3-benzothiadiazine-1,1-dioxide, 3-chloro-4-methyl-4H-benzo[e][1,2,4]thiadiazine-1,1-dioxide, and 3-chloro-4-ethyl-4H-benzo[e][1,2,4]thiadiazine-1,1-dioxide. In some embodiments, the acridinethione is substituted by one or more amino or alkoxy group. In some embodiments, the acridinethione is selected from the group consisting of 3-amino-10H-acridone-9-thione (3ATA), 9(10H)-acridinethione, 1,4-dimethoxy-10H-acridine-9-thione, and 2,2′-diphenyldiamine-bis-[N,N′-[3-amido-N-methylamino)-10H-acridine-9-thione]].

In some embodiments, the subject has been exposed to ionizing radiation, will be exposed to ionizing radiation, or is at risk of incurring exposure to ionizing radiation as the result of radiological agent exposure during warfare, a radiological terrorist attack, an industrial accident, or space travel. Subjects can further be exposed to, or be scheduled to be exposed to, ionizing radiation when undergoing therapeutic irradiation for the treatment of proliferative disorders. Such disorders include cancerous and non-cancer proliferative diseases. For example, the presently disclosed compounds are believed effective in protecting healthy hematopoietic stem/progenitor cells during therapeutic irradiation of a broad range of tumor types, including but not limited to the following: breast, prostate, ovarian, skin, lung, colorectal, brain (i.e., glioma) and renal. Ideally, growth of the cancer being treated by IR should not be affected by the selective CDK 4/6 inhibitor. As would be understood by one of skill in the art, the potential sensitivity of certain tumors to CDK4/6 inhibition can be deduced based on tumor type and molecular genetics. Cancers that are not expected to be affected by the inhibition of CDK4/6 are those that can be characterized by one or more of the group including, but not limited to, increased activity of CDK1 or CDK2, loss or absence of retinoblastoma (RB) tumor suppressor protein, high levels of MYC expression, increased cyclin E and increased cyclin A. Such cancers can include, but are not limited to, small cell lung cancer, retinoblastoma, HPV positive malignancies like cervical cancer and certain head and neck cancers, MYC amplified tumors such as Burkitts Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancer and certain classes of testicular cancers.

For example, in some embodiments, the cancer is selected from a small cell lung cancer, retinoblastoma and triple negative (ER/PR/Her2 negative) or “basal-like” breast cancer. Small cell lung cancer and retinoblastoma almost always inactivate the RB tumor suppressor protein, and therefore do not require CDK4/6 activity to proliferate. Thus, CDK4/6 inhibitor treatment will effect PQ in the bone marrow and other normal host cells, but not in the tumor. Triple negative (basal-like) breast cancer is also almost always RB-null. Also, certain virally induced cancers (e.g. cervical cancer and subsets of Head and Neck cancer) express a viral protein (E7) which inactivates the RB protein making these tumors functionally RB-null. Some lung cancers are also believed to be caused by HPV. As would be understood by one of skill in the art, cancers that are not expected to be affected by CDK4/6 inhibitors (e.g., those that are RB-null, that express viral protein E7, or that overexpress MYC) can be determined through methods including, but not limited to, DNA analysis, immunostaining, Western blot analysis, and gene expression profiling.

Selective CDK4/6 inhibitors are also believed useful in protecting healthy hematopoietic stem/progenitor cells during therapeutic irradiation of abnormal tissues in non-cancer proliferative diseases, including but not limited to the following: hemangiomatosis in newborns, secondary progressive multiple sclerosis, chronic progressive myelodegenerative disease, neurofibromatosis, ganglioneuromatosis, keloid formation, Paget's Disease of the bone, fibrocystic disease of the breast, Peronies and Duputren's fibrosis, restenosis and cirrhosis.

According to the presently disclosed subject matter, therapeutic ionizing radiation can be administered to a subject on any schedule and in any dose consistent with the prescribed course of treatment, as long as the radioprotectant/radiomitigant compound is administered prior to, during, or following the radiation. Generally, the radioprotectant and/or radiomitigant compound is administered to the subject during the time period ranging from 24 hours prior to radiation exposure until 24 hours following radiation exposure. However, this time period can be extended to time earlier that 24 hour prior to exposure to the radiation (e.g., based upon the time it takes the compound to achieve suitable plasma concentrations and/or the compounds plasma half-life). Further, the time period can be extended longer than 24 hours following exposure to the radiation so long as later administration of the compound leads to at least some protective effect. If desired, multiple doses of the radioprotectant compound can be administered to the subject. Alternatively, the subject can be given a single dose of the inhibitor. The course of treatment differs from subject to subject, and those of ordinary skill in the art can readily determine the appropriate dose and schedule of therapeutic radiation in a given clinical situation.

### III. Active Compounds, Salts and Formulations

As used herein, the term “active compound” refers to a selective CDK 4/6 inhibitor compound or a pharmaceutically acceptable salt thereof. The active compound can be administered to the subject through any suitable approach. The amount and timing of active compound administered can, of course, be dependent on the subject being treated, on the dosage of IR to which the subject has been, or is anticipated of being exposed to, on the manner of administration, on the pharmacokinetic properties of the active compound, and on the judgment of the prescribing physician. Thus, because of subject to subject variability, the dosages given below are a guideline and the physician can titrate doses of the compound to achieve the treatment that the physician considers appropriate for the subject. In considering the degree of treatment desired, the physician can balance a variety of factors such as age and weight of the subject, presence of preexisting disease, as well as presence of other diseases. Pharmaceutical formulations can be prepared for any desired route of administration including, but not limited to, oral, intravenous, or aerosol administration, as discussed in greater detail below.

The therapeutically effective dosage of any specific active compound, the use of which is within the scope of embodiments described herein, can vary somewhat from compound to compound, and subject to subject, and can depend upon the condition of the subject and the route of delivery. As a general proposition, a dosage from about 0.1 to about 200 mg/kg can have therapeutic efficacy, with all weights being calculated based upon the weight of the active compound, including the cases where a salt is employed. In some embodiments, the dosage can be the amount of compound needed to provide a serum concentration of the active compound of up to between about 1 and 5 μM. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. In some embodiments, dosages can be from about 1 μmol/kg to about 50 μmol/kg, or, optionally, between about 22 μmol/kg and about 33 μmol/kg of the compound for intravenous or oral administration.

In accordance with the presently disclosed methods, pharmaceutically active compounds as described herein can be administered orally as a solid or as a liquid, or can be administered intramuscularly, intravenously or by inhalation as a solution, suspension, or emulsion. In some embodiments, the compounds or salts also can be administered by inhalation, intravenously, or intramuscularly as a liposomal suspension. When administered through inhalation the active compound or salt can be in the form of a plurality of solid particles or droplets having a particle size from about 0.5 to about 5 microns, and optionally from about 1 to about 2 microns.

The pharmaceutical formulations can comprise an active compound described herein or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

In addition to the active compounds or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulation is placed in a vial designed for multi-dose use. The pharmaceutical formulations described herein can be lyophilized using techniques well known in the art.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed subject matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the subject matter described herein, there is provided an injectable, stable, sterile formulation comprising an active compound as described herein, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, and optionally from about 0.5 to about 5 microns. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation can comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used herein, the term “water-soluble” is meant to define any composition that is soluble in water in an amount of about 50 mg/mL, or greater. Also, as used herein, the term “water-insoluble” is meant to define any composition that has a solubility in water of less than about 20 mg/mL. In some embodiments, water-soluble compounds or salts can be desirable whereas in other embodiments water-insoluble compounds or salts likewise can be desirable.

The term “pharmaceutically acceptable salts” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the presently disclosed subject matter.

Thus, the term “salts” refers to the relatively non-toxic, inorganic and organic acid addition salts of compounds of the presently disclosed subject matter. These salts can be prepared in situ during the final isolation and purification of the compounds or by separately reacting the purified compound in its free base form with a suitable organic or inorganic acid and isolating the salt thus formed. In so far as the compounds of the presently disclosed subject matter are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the presently disclosed subject matter.

Pharmaceutically acceptable base addition salts are formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine.

The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the presently disclosed subject matter.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

### EXAMPLES

The following Examples provide illustrative embodiments. In light of the present disclosure and the general level of skill in the art, those of skill can appreciate that the following Examples are intended to be exemplary only and that numerous changes, modifications, and alterations can be employed without departing from the scope of the presently disclosed subject matter.

### Methods

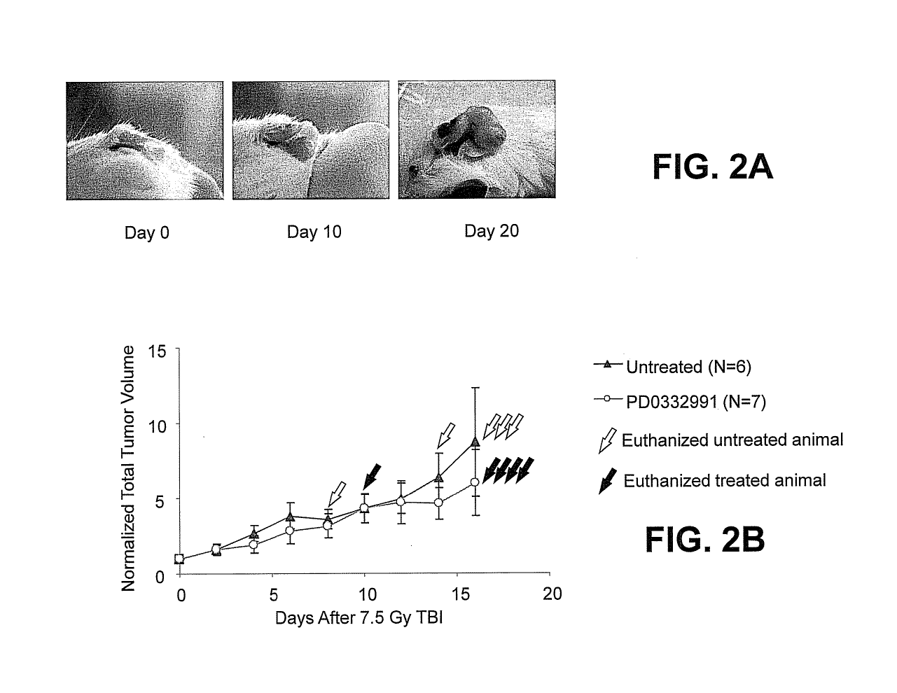

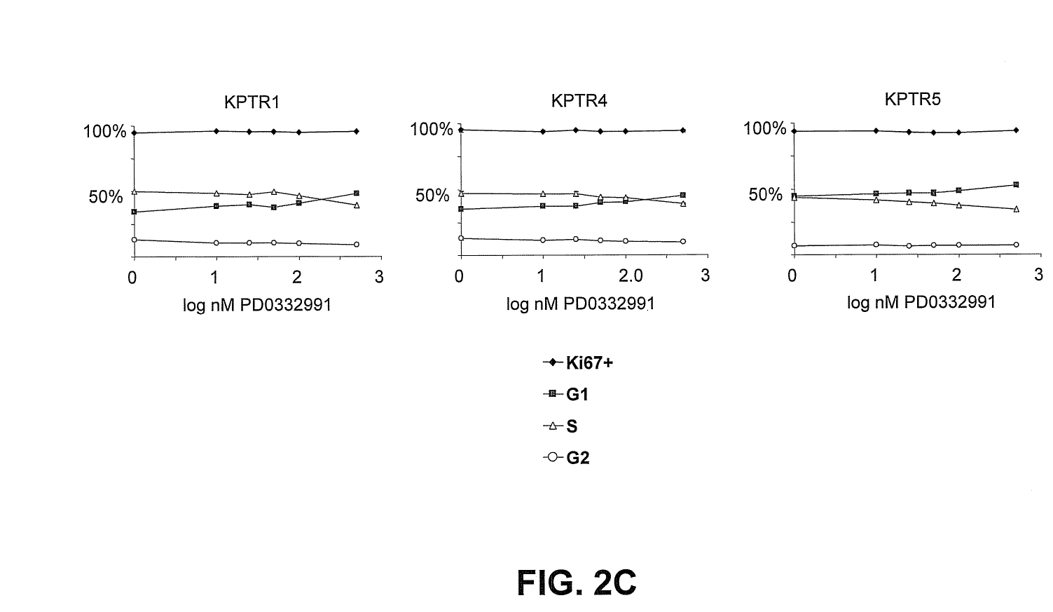

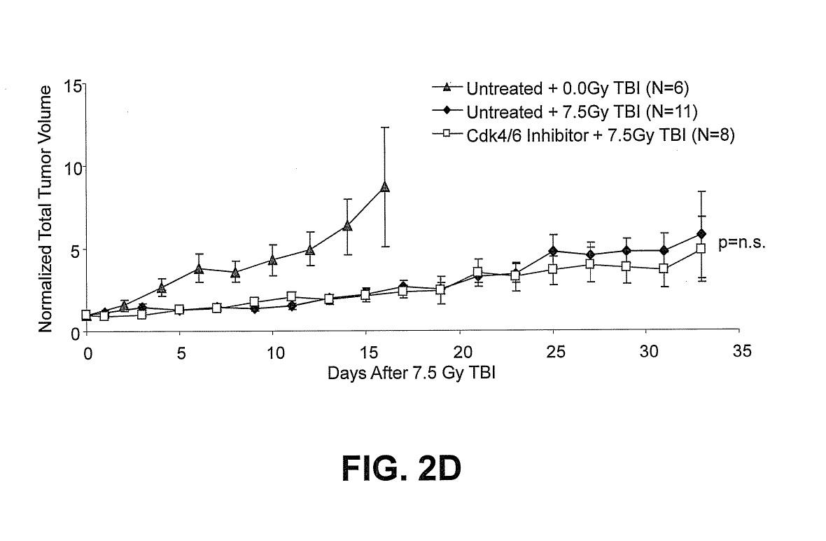

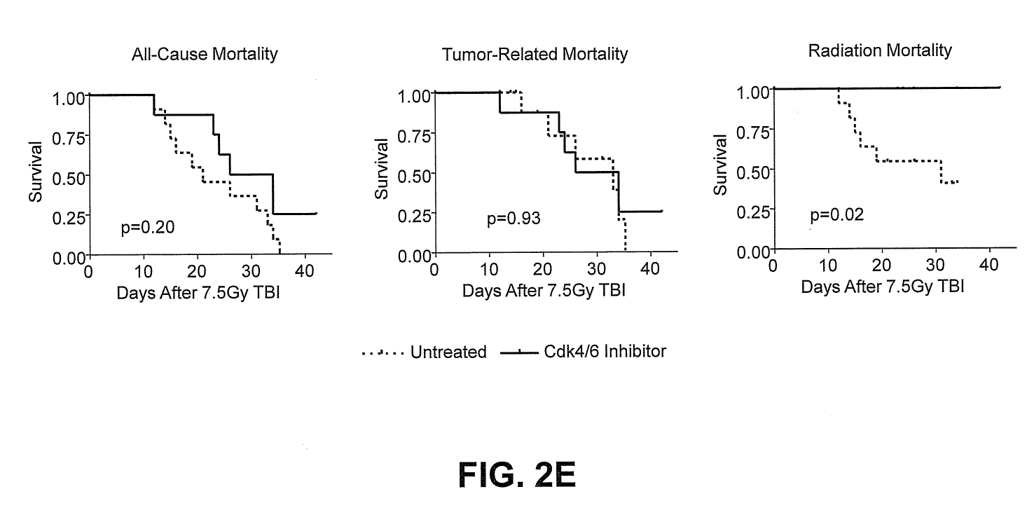

Animals: All mice were housed in accord with the University of North Carolina at Chapel Hill (UNC-CH) Institutional Animal Care and Use Committee. Young adult (6-12 weeks of age), virgin female C57B1/6 mice were purchased from Jackson Labs (Bar Harbor, Me., United States of America). C3H mice were obtained from Harlan Sprague-Dawley Inc. (Indianapolis, Ind., United States of America). Experiments on tumor-bearing TyrRAS+Ink4a/Arf−/− mice (see Chin et al., Genes & development, 11, 2822-2834 (1997)) were performed in animals fully backcrossed (N>10) to the FVB/n background. Mice were treated as previously described (see Ramsey et al., Cancer Res., 67, 4732-4741 (2007)) with PD 0332991 obtained from Pfizer Inc., (New York, N.Y., United States of America) given by oral gavage at a dose of 150 mg/kg body weight. TyrRAS+Ink4a/Arf−/− mice were serially observed until tumor development. When tumors were about 0.2 cm² in size, daily PD 0332991 treatment was begun. The data shown in FIGS. 2B and 2D were normalized to tumor size at the time of therapy initiation. Tumor-bearing mice were euthanized at the indicated times for morbidity, tumor ulceration, or tumor size >1.5 cm in diameter.

For TBI experiments, mice were irradiated using a ¹³⁷Cs source (AECL Gammacell 40 Irradiator, Atomic Energy of Canada Ltd, Mississauga, Ontario, Canada). Delivered doses were at or near the empirically determined LD₉₀ of 7.5 Gy, consistent with prior studies. See Na Nakorn et al., J. Clin. Invest., 109, 1579-1585 (2002); Herodin et al., Blood, 101, 2609-2616 (2003); Uckun et al., Blood, 75, 638-645 (1990); and Wang et al., Proc. Natl. Acad. Sci., USA, 94, 14590-14595 (1997). Peripheral blood was collected using a tail vein nick for complete blood cells (CBCs) and analyzed with a HemaTrue analyzer (Heska Co., Loveland, Colo., United States of America).

Cell Lines: KPTR1, KPTR4. and KPTR5 were derived from tumor-bearing TyrRAS+Ink4a/Arf−/− by standard procedures and cultured in RPMI+10% fetal bovine serum (FBS). Telomerized HDFs (tHDFs, also known as HS68) were cultured in DMEM+10% FBS with penicillin and streptomycin. The same conditions were used for A2058 and WM2664, human melanoma cell lines with known RB-pathway mutations: A2058 is RB-null, whereas WM2664 lacks p16^{INK4a}/Arf. See Shields, et al., Cancer Res., 67, 1502-1512 (2007). Cells were irradiated for indicated doses in a Rad Source Inc. (Alpharetta, Ga., United States of America) RS-2000 Biological Irradiator at 160 kV, 25.0 mA, at distance setting 1, with a dose rate of 103 rads/min.

BrdU Incorporation in vitro: Cells were plated and allowed to adhere overnight or at least 6 hours prior to adding CDK4/6 inhibitor at the indicated concentrations. Cells were grown for 24 hours in the presence of CDK4/6 inhibitor. 15 minutes prior to cell harvesting, 5-Bromo-2-deoxyuridine (BrdU) was added to the media at a final concentration of 10 μM. The cells were then washed, trypsinized, pelleted, fixed, permeabilized, stained, and measured on a flow cytometer as described in manufacturer directions for the BrdU Kit (BD Biosciences Pharmingen, San Diego, Calif., United States of America).

BrdU Incorporation in vivo: Mice were treated once daily for 2 days with PD 0332991 at 150 mg/kg. 5-Bromo-2-deoxyuridine (BrdU) was given 24 hours after the initiation of PD 0332991 and continued until the animals were sacrificed for analysis: 24 hours of BrdU by intraperitoneal (i.p.) injection every 6 hours at a dose of 1 mg.

Bone Marrow Immunophenotyping and Proliferation by Flow Cytometry:

For HSPC proliferation experiments, mice received daily oral gavage with PD0332991 for 2 days with 1 mg BrdU intraperitoneal injection every 6 hours for 24 hours prior to sacrifice. BM-MNC harvest and immunophenotyping was performed using RBC lysis, biotin-conjugated Lin-panel incubation (Invitrogen Corporation, Carlsbad, Calif., United States of America), paramagnetic bead-conjugated straptavidin (Miltinyi Biotec, Bergisch Gladbach, Germany) incubation, and magnetic depletion using an AutoMACS (Miltinyi Biotec, Bergisch Gladbach, Germany). At least 2×10⁶ Lin-depleted cells per mouse were incubated with fluorescently labeled antibodies against cell surface antigens used to identify hematopoietic progenitor subpopulations as previously described (see Passegue et al., J. Exp. Med., 202, 1599-1611 (2005); and Kiel et al., Cell, 121, 1109-1121 (2005): CD34-FITC, CD16/32-PacificBlue, IL7Ra-PE-Cy5, and cKit-APC-Alexa750 from eBiosciences, Inc. (San Diego, Calif., United States of America); Sca1-PE-Cy7, CD150-PE-Cy5, and CD48-PacificBlue from BioLegend (San Diego, Calif., United States of America); and Aqua Live/Dead viability dye (Invitrogen Corporation, Carlsbad, Calif., United States of America). Streptavidin-PE-TexasRed (Invitrogen Corporation, Carlsbad, Calif., United States of America) was used to confirm efficiency of lineage-depletion. After cell surface staining, cells were fixed, permeabilized, and stained with antibodies against Ki67-FITC, BrdU-APC, and caspase3-PE (BD Biosciences Pharmingen, San Diego, Calif., United States of America).

In all experiments, gating based on isotype controls was used as appropriate. Flow cytometry was performed using a CyAn ADP (Dako, Glostrup, Denmark) and analyzed with FlowJo software (Tree Star, Ashland, Oreg., United States of Ameria). For each bone marrow sample, a minimum of 200,000 cells were analyzed. For cell culture samples, a minimum of 20,000 cells were analyzed.

Mass Spectrometry: Mice were dosed as described and tail vein nick performed to acquire 30 μL periphal blood. Blood was centrifuged to acquire 10 μL plasma, which was mixed with 100 μL ice cold methanol, centrifuged, and mixed with 10 μL of an internal control. Plasma levels were quantified based on standard curves derived in triplicate from known concentrations of wild type C57Bl/6 female mouse plasma unexposed to the measured drug. Quantification was performed using HPLC separation with triple quadrupole mass spectrometry normalizing results to measurement of the internal standard.

Cobblestone Area-forming Cell (CAFC) Assay: The CAFC assay was performed as described previously. See Meng et al., Cancer Res., 63, 5414-5419 (2003). Briefly, bone marrow was harvested from femurs and tibias of mice and centrifuged to purify BM-MNCs. The frequencies of CAFC were determined at weekly intervals (on day 7, 14, and 35). Wells were scored positive if at least one phase-dark hematopoietic clone (containing 5 or more cells) was seen. The frequency of CAFC was then calculated by using Poisson statistics as described.

γH2AX after IR: For γH2AX images, tHDF received 6 Gy IR with or without 24 hours of prior exposure to a CDK inhibitor. Immediately or 3 hours after IR exposure, cells were washed 2× with ice cold PBS, fixed with 4% paraformaldehyde+0.1% Triton-X (Sigma, St Louis, Mo., United States of America) for 30 min, washed 2× with ice cold PBS, incubated with anti-γH2AX-AlexaFluor488 (Cell Signaling Technology, Beverly, Mass., United States of America) and phalloidin-AlexaFluor568 (Invitrogen Corporation, Carlsbad, Calif., United States of America) for 30 minutes, washed 4× with ice cold phosphate buffered saline (PBS), and mounted. Images were obtained by fluorescent microscopy (see Microscopy). Images were analyzed for mean nuclear intensities were computed in ImageJ software (developed by the National Institutes of Health, Bethesda, Md., United States of America). For flow cytometry γH2AX data, cells were incubated with a CDK inhibitor for 24 hours prior to exposure to 0, 2, 4, 6, or 8 Gy of ionizing radiation. After irradiation cells were immediately fixed and stained for γH2AX according to the instructions in γH2AX Flow Kit from Millipore (Billerica, Mass., United States of America).

Clonogenic Assay: The clonogenic assay was performed as previously described (see Franken et al., Nature protocols, 1, 2315-1319 (2006)) with cells plated in a six-well plate at least 6 hours prior to treatment with CDK inhibitor for 24 hours, with 6 Gy IR occurring 12 hours after initiation of CDK inhibitor exposure. Cells were grown for 16 days, washed, fixed, and stained with crystal violet. Plates were then imaged using an Odyssey infrared scanner (Li-Cor Biosciences, Lincoln, Nebr., United States of America) and quantified using the accompanying software.

Comet tail assay: Cells were seeded and allowed to adhere overnight in a 6 cm culture dish. Cells were then treated for 24 hours with a CDK4/6 inihibitor or dimethyl sulfoxide (DMSO) only. After 24 hours cells were irradiated as described. Cells were then fixed and treated as described in manufacturer instructions for the COMETASSAY™ purchased from Trevigen (Gaithersburg, Md., United States of America). In summary, cells were embedded in agarose, the membranes are permeabilized, DNA is denatured in alkali solution, and the DNA is mobilized using gel electrophoresis. Images of the comet tails were obtained by fluorescent microscopy (see Microscopy) and images were analyzed using CometScore software from TriTek Corp. (Sumerduck, Va., United States of America) at either 10× or 20× magnification.

Microscopy: Micrographs were obtained using a mercury laser attached to an inverted microscope (model IX-81, Olympus, Center Valley, Pa., United States of America) equipped with a 10 PlanApo objective, 20 PlanApo objective, or 40 PlanApo objective attached to a CCD camera (model C4742-80-12AG, OCAR-ER, Hamamatsu Corporation, Hamamatsu City, Japan), and controlled by Slidebook software.

Western Blots: Western blots were performed on cell lysates in NP-40 lysis buffer with protease inhibitors (Roche, Basel, Switzerland) and phosphatase inhibitors (Calbiochem, San Diego, Calif., United States of America) as previously described (see Ramsey et al., Cancer Res., 67, 4732-4741), using anti-p53-phospho-Ser15 (Cell Signaling Technology, Beverly, Mass., United States of America), Bax, p21, and actin-HRP (Santa Cruz Biotechnology, Inc., Santa Cruz, Calif., United States of America).

Compounds: The compounds used in the following studies are shown in Table 1, below. Unless otherwise noted, the compounds were freshly synthesized via known literature routes (see e.g., Chu et al., J. Med. Chem, 49, 6549-6560 (2006); Zhu et al., J. Med. Chem. 46, 2027-2030 (2003), Toogood et al., J. Med. Chem., 48, 2388-2406 (2005); and Fry et al., Mo. Cancer Ther., 3, 1427-1438 (2004) or purchased from commercial sources. Flavopiridol was provided by Dr. Kwok-Kin Wong (Dana-Farber Cancer Institute, Harvard Medical School, Boston, Mass., United States of America). Roscovitine and genistein were purchased from LC Laboratories (Woburn, Mass., United States of America). 2BrIC was freshly synthesized for use in the present studies by OTAVA Chemicals (Kiev, Ukraine), but is also commercially available from OTAVA Chemicals (Kiev, Ukraine) and Alexis Biocemicals (EnzoLife Sciences, Inc., Farmingdale, N.Y., United States of America). PD0332991 was provided by Pfizer, Inc. (New York, N.Y., United States of America) or was synthesized as described below in Example 6. The structure and purity of all compounds was confirmed by NMR and LC-MS. All compounds were >94% pure.

TABLE 1
Selective and Non-Selective CDK4/6 Inhibitor Compounds.

| Compound | Structure |
|---|---|
| 1 | 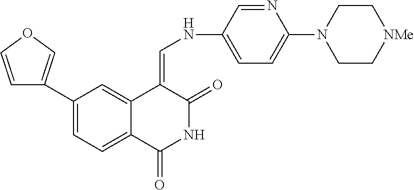 |
| 2 | 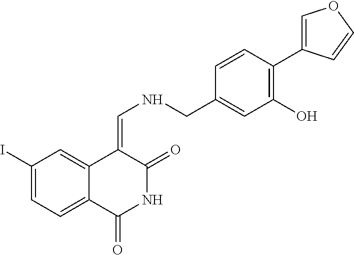 |
| 3 | 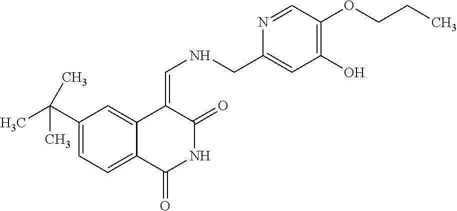 |
| 4 | 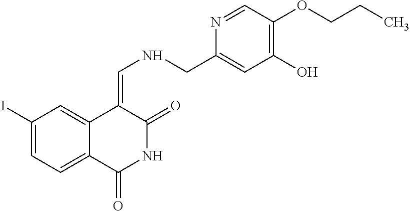 |
| 2BrIC | 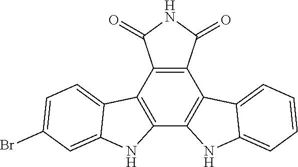 |
| 5 | 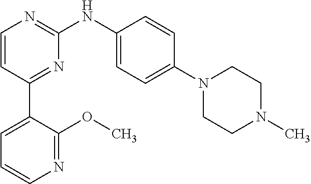 |
| 6 | 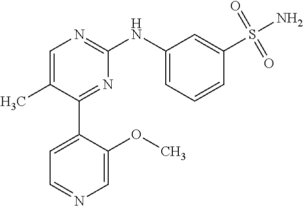 |
| 7 (R547) | 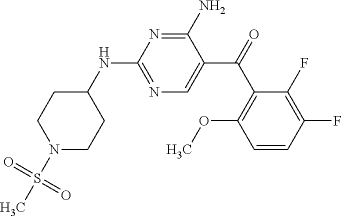 |
| PD (PD0332991) | 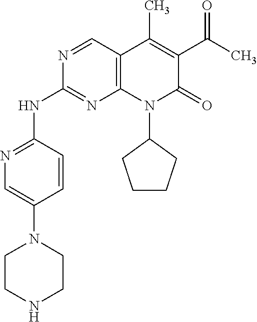 |
| flavopiridol | 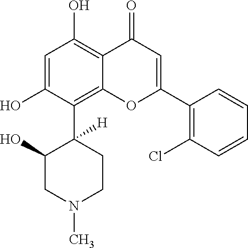 |
| Roscovitine | 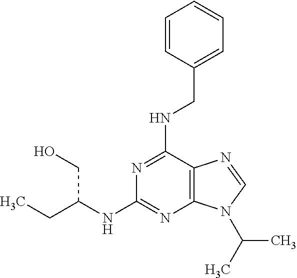 |
| Genistein | 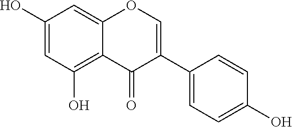 |
| 8 | 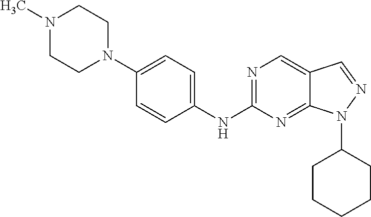 |
| 9 | 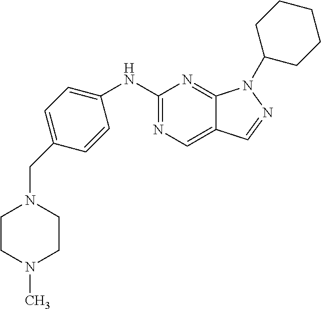 |
| 10 | 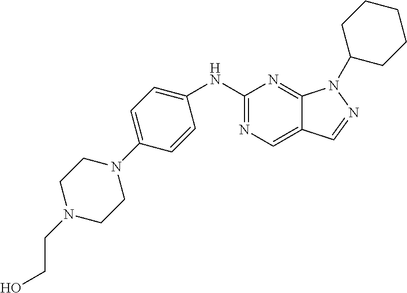 |
| 11 | 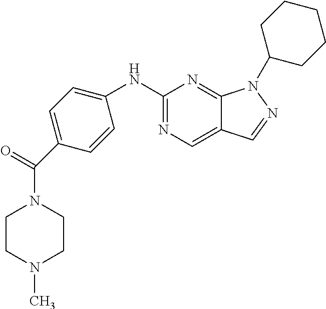 |
| 12 | 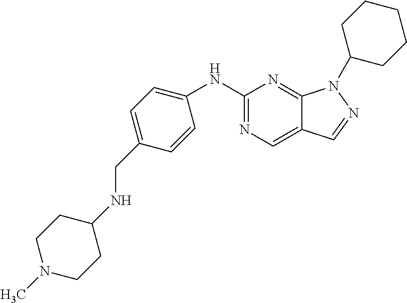 |
| 13 | 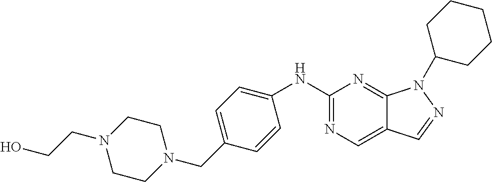 |
| 14 | 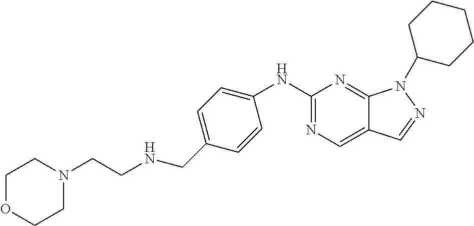 |
| 15 | 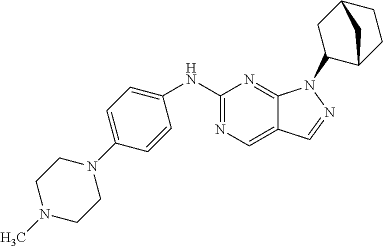 |

Statistical Analysis: Unless otherwise noted, comparisons are made with one-way ANOVA with Bonferroni correction for multiple comparisons where appropriate. Error bars are +/−standard error of the mean (SEM).

### Example 1

### In Vivo Activity in Genetically Engineered Murine Melanoma Model

It has been suggested that melanoma appears likely to require persistent CDK4/6 activity for tumor maintenance because cyclin D1 is a major target of the RAS-RAF-ERK pathway, which is activated in the vast majority of melanoma (see Curtin et al., N. Engl. J. Med., 353, 2135-2147 (2005)) and somatic p16^{INK4a} inactivation is seen in the majority of human melanoma. See Walker et al., Genes Chromosomes Cancer, 22, 157-163 (1998); and Daniotti et al., Oncogene, 23, 5968-5977 (2004). Therefore, this tumor type is characterized by two genetic lesions that would be expected to activate CDK4 and/or CDK6. In order to study the role of CDK4/6 activity in melanoma maintenance, the well characterized Tyr-RAS+INK4a/Arf−/− model of melanoma developed by Chin and co-workers was used in the FVB/n genetic background. See Chin et al., Genes & Development, 11, 2822-2834 (1997). In this genetically engineered murine model (GEMM), melanocyte-specific expression of mutant H-Ras induces progressive melanoma in the setting of loss of the p16^{INK4a} and An tumor suppressor proteins (Ink4a/Arf−/−). See FIGS. 2A and 2B. Although mice harboring the transgene in the setting of intact Ink4a/Arf function are phenotypically normal and do not develop melanoma spontaneously, specific loss of p16^{INK4a} (with preserved Arf function) accelerates tumor formation (see Sharpless et al., Oncogene, 22, 5055-5059 (2003)), which, without being bound to any one theory, suggests that CDK4/6 activation is crucial for tumor initiation.

To study the role of CDK4/6 activity in melanoma maintenance, tumor-bearing Try-RAS+Ink4aArf−/− animals were treated with 150 mg/kg of PD 0332991 by oral gavage daily. This dose and schedule can be well tolerated without evident toxicity (see Fry et al., Mol. Cancer Ther., 3, 1427-1438 (2004); and Ramsey et al., Cancer Res., 67, 4732-4741 (2007)); but no regression or decrease in tumor growth was noted in PD 0332991 treated mice. See FIG. 2B. These animals were treated with a maximally tolerated dose on a schedule that has potent activity in some xenograft systems. See Fry et al., Mol. Cancer Ther., 3, 1427-1438 (2004). Not only was no effect seen on the growth of established tumors, but two of five mice developed new tumors in the 8-16 days while receiving PD 0332991 treatment. Tumor growth did not appear to be due to insufficient bioavailability of PD 0332991, because the same dose and schedule abrogated proliferation in CDK4/6 dependent normal murine tissues such as the pancreatic islet. See Ramsey et al., Cancer Res., 67, 4732-4741 (2007). Similarly, derivative cell lines from these tumors were insensitive to PD 0332991 treatment in vitro. See FIG. 2C. Therefore, even through p16INK4a loss facilitates initiation of this tumor type in this model (see Sharpless et al., Oncogene, 22, 5055-5059 (2003)), established RAS-driven murine melanomas of this type do not require CDK4/6 activity for growth in vivo, suggesting that cancers can rely on the activity of another proliferative kinase (e.g., CDK 2 or CDK1) for proliferation.

Given that these results establish that these RAS-induced murine melanomas are not dependent on CDK4/6 activity, the effects of CDK4/6 treatment 4 hours prior to an administration of therapeutic IR (7.5) Gy was tested (See FIGS. 2D and 2E). Pre-treatment with CDK4/6 inhibitor prior to IR did not compromise the anti-cancer efficacy of IR in this model, but did decrease radiation-associated toxic death. Therefore, PQ induction through the use of selective CDK4/6 inhibitors can prevent the hematologic toxicity of IR without necessarily compromising the efficacy of therapeutic IR, at least in cancers that do not require CDK4/6 activity for proliferation.

### Example 2

### Selective G1 Arrest in CDK4/6-Dependent Cells

Several human cell lines were exposed to the selective and nonselective small molecule CDK inhibitors shown in Table 1, above. CDK4/6 dependent cell lines, including telomerized human diploid fibroblasts (tHDF) and human melanoma cell line WM2664, demonstrated strong, reversible G1-arrest after exposure to the potent and selective Cdk4/6 inhibitors PD0332991 or 2BrIC. See FIGS. 3A-3B. In contrast, less selective CDK inhibitors, such as those that additionally target CDK1/2, including roscovitine, compound 7 (i.e., R547), and flavopiridol variably produced a G2/M block, intra-S arrest, or cell death (sub-GO) in these cell types. Compounds 1-6 and 8-15 from Table 1 also showed lack of selective G1 arrest. An RB-null melanoma line, A2058, was, as expected, insensitive to selective CDK4/6 inhibition, but similarly displayed a G2/M or intra-S arrest and/or cell death after exposure to the less specific CDK inhibitors. The proliferation of seven RB-deficient human small cell lung cancer lines was also resistant to the selective CDK4/6 inhibitors. Thus, the presently disclosed data indicates that structurally distinct, potent and selective Cdk4/6 inhibitors affect a substantially pure (i.e. “clean”) G1-arrest in susceptible cell lines (CDK4/6-dependent cell lines), whereas the cell cycle effects of more global, nonspecific CDK inhibitors are less predictable and associated with cytotoxicity.

### Example 3

### Protection from IR-Induced DNA Damage in Cells

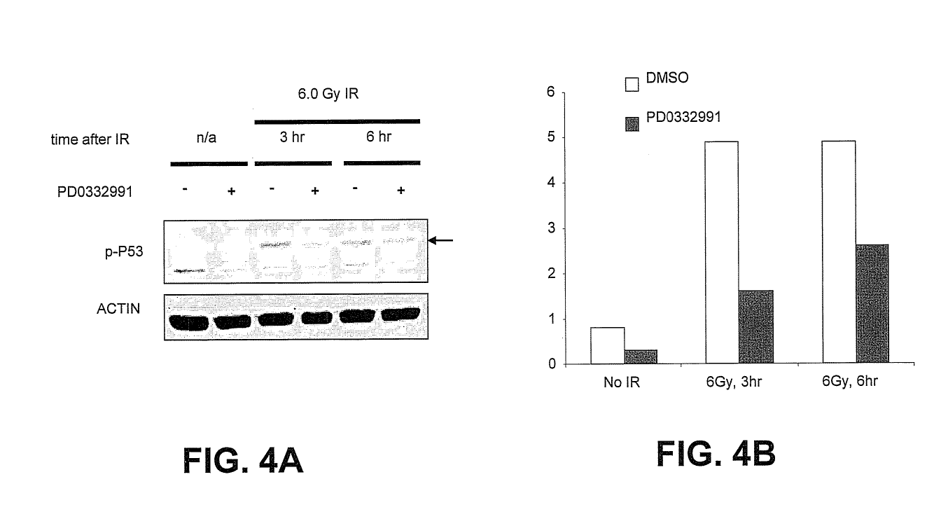

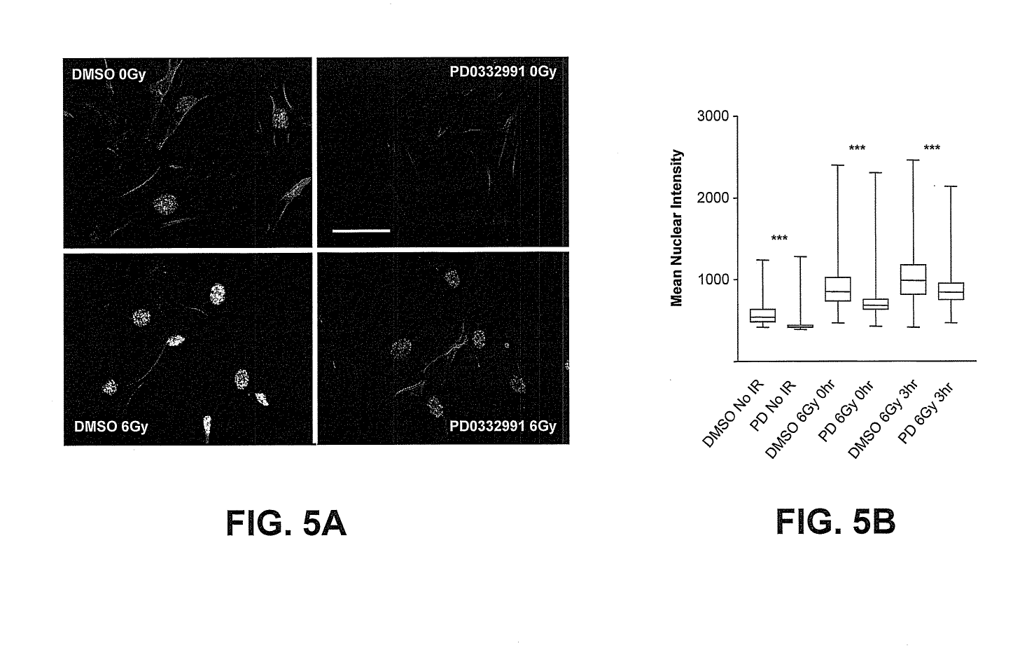

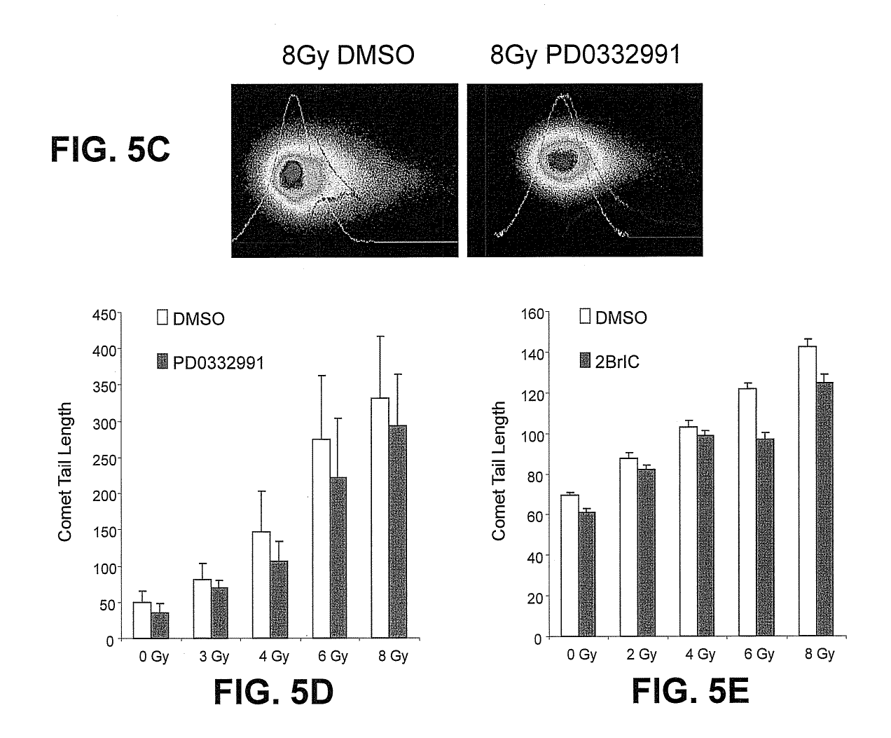

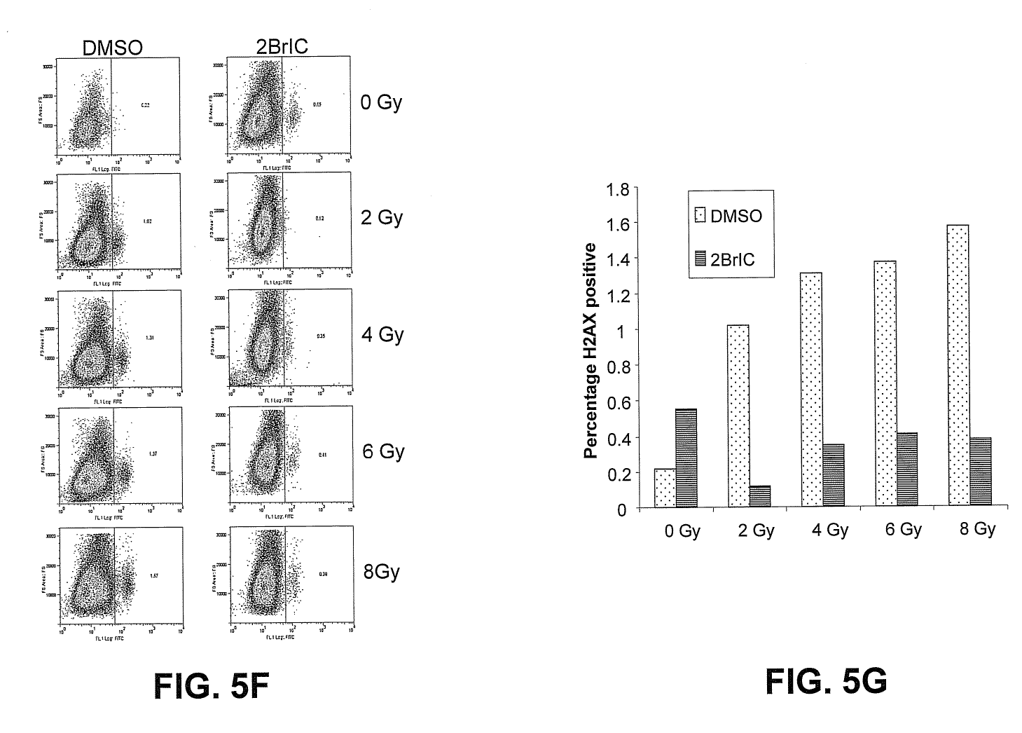

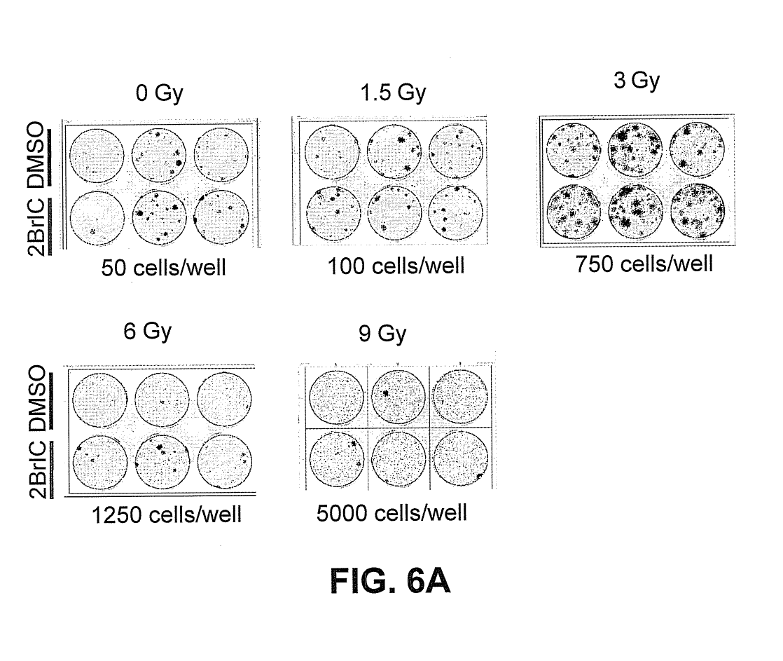

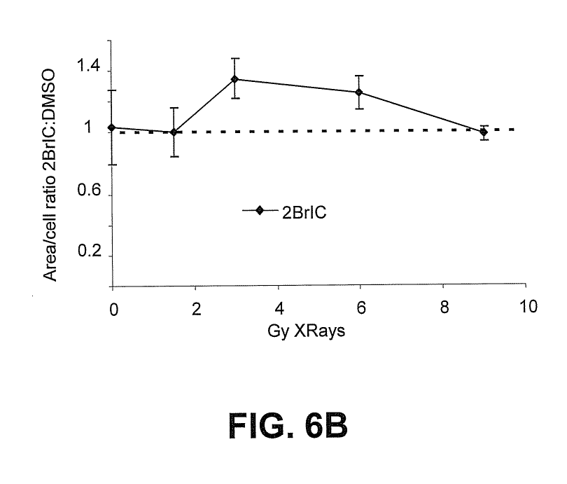

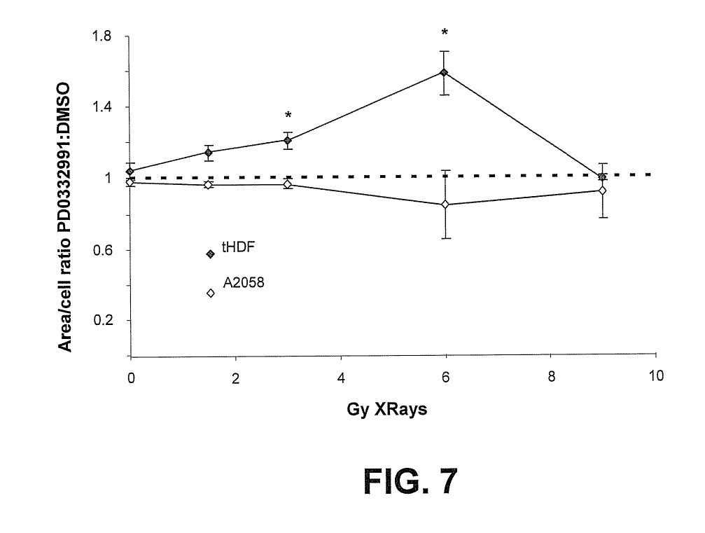

IR exposure caused extensive DNA damage (comet tails and γH2AX foci) and DNA damage response (p53 expression) in all cell lines tested, including CDK4/6 dependent cell lines. Treatment with selective CDK4/6 inhibitors PD0332991 or 2BrIC prior to IR attenuated DNA damage response (see FIGS. 4A-4B), γH2AX formation (see FIGS. 5A, 5B, 5F, and 5G), and DNA damage (comet tails; see FIGS. 5C-5E) only in cell lines in which CDK4/6 inhibition causes a clean G1-arrest. For example, telomerized human diploid fibroblast (tHDF) cells were either pretreated with 100 nM PD0332991 for 24 hours and then exposed to 6 Gy IR, exposed to 6 Gy IR without PD0332991 pretreatment, or simply treated with PD0332991 but not exposed to IR. The cells were then stained for γH2AX foci (green) and phalloidin (red). Strong green nuclear patches, indicating DNA damage (i.e., γH2A)(foci), were present in the IR-only treated tHDFs, but were not substantially present in PD0332991-treated cell samples, indicating that pretreatment with PD 0332991 was able to protect the cells from DNA damage caused by IR (shown in gray scale in FIG. 5A and quantified in FIG. 5B). A similar protection from IR-induced γH2AX foci formation was afforded by treatment with 2BrIC. See FIGS. 5F-5G. Likewise, PD0332991 and 2BrIC attenuated IR-induced DNA damage in a comet tail assay, a direct measurement of DNA damage. See FIGS. 5C-5E. Furthermore, pretreatment with these compounds prior to IR exposure provided enhanced clonogenic cell survival in a CDK4/6 dependent manner. See FIGS. 6A-6B and FIG. 7.

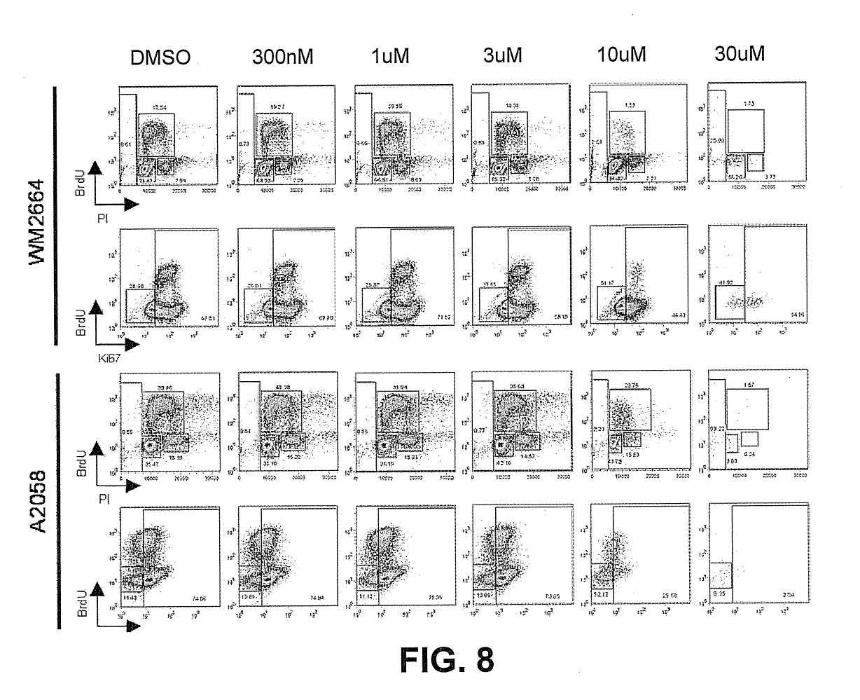

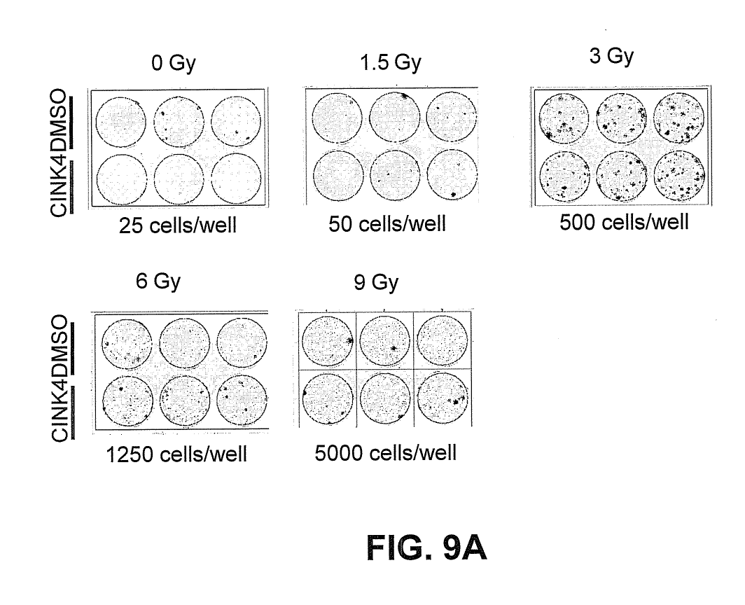

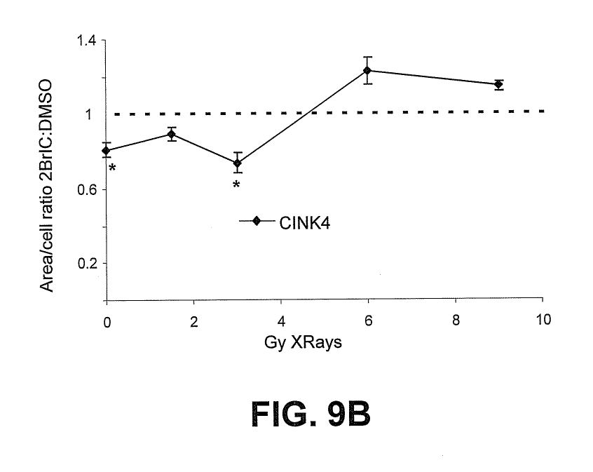

In contrast, CINK4, a nonselective CDK4/6 inhibitor, did not induce a substantially pure (i.e. “clean”) G1 arrest in CDK4/6-dependent (WM2664) or independent (A2058) cells (see FIG. 8) at concentrations between 300 nM and 30 μM, and CINK4 failed to enhance cell survival following IR exposure. See FIGS. 9A-9B. Other non-selective CDK inhibitors also failed to afford cellular protection from IR, with some agents increasing IR sensitivity (e.g. staurosporine) in some CDK4/6-dependent cell types. The failure of less selective CDK inhibitors to afford protective PQ suggests that arrest in a phase of the cell cycle other than G1 (e.g. G2/M) might not protect from genotoxic exposure. Alternatively, it is also possible the less selective CDK inhibitors prevent phosphorylation of non-RB family substrates (e.g. BRCA1 by CDK1/2) and thereby untowardly augment the toxicity of DNA damaging agents as suggested by the observed CINK4 effect in FIGS. 9A-9B. Together, these data show that PQ effected by selective CDK4/6 inhibitors, but not more global CDK inhibitors, provides in vitro resistance to IR-induced DNA damage in cell types that require CDK4/6 kinase activity for G1 to S traversal.

### Example 4

### Protection of Hematopoietic Stem Cells from IR In Vivo

The presently disclosed in vitro data of Examples 2 and 3 suggested that CDK4/6-dependent tissues could also be protected from IR-induced DNA damage by CDK4/6 inhibitors in vivo. PD0332991, which is orally bioavailable, was administered to adult wild-type C57Bl/6 mice by oral gavage. Proliferation of hematopoietic stem cells (HSC; Lin−Kit+Sca1+CD48−CD150+) measured by Ki67 expression and incorporation of bromodeoxyuridine (BrdU) over 24 hours (see FIGS. 10B-10C) was slow, comparable to prior estimates. See Passeque et al., J. Exp. Med., 202, 1599-1611 (2005); Wilson et al., Cell, 125, 1118-1129 (2008); and Kiel et al., Nature, 449, 238-U210 (2007). PD0332991 treatment for 48 hours significantly decreased the frequency of HSC doubly positive for expression of Ki67 and BrdU (see FIGS. 10B-10C), with more pronounced effects on Ki67 expression. A more pronounced inhibition of proliferation was noted in the more rapidly proliferating multipotent progenitor cell compartment (MPP; Lin−Kit+Sca1+CD48−CD150−). See FIGS. 10B-10C. Oligopotent progenitors (Lin−Kit+Sca1−) demonstrated modest inhibition of proliferation (see FIGS. 10B,C), with the strongest effects seen in common myeloid progenitors (CMP) and common lymphocyte progenitors (CLP) compared to weaker effects in the more differentiated granulocyte-monocyte progenitors (GMP) and megakaryocyte-erythroid progenitors (MEP). See FIGS. 10B-10C. In contrast to these effects on early HSPC, no change in proliferation was noted in the more fully differentiated Lin−Kit−Sca1− and Lin+ cells, though these fractions are heterogeneous and effects on subpopulations can be obscured.

2BrIC was solubilized for oral gavage using formulation #6 from the Hot Rod formulation kit (Pharmatek, Inc. San Diego, Calif., United States of America) and given by oral gavage 2 hours prior to BrdU injection. Also, an additional dose was given at the time of BrdU injection to sustain the G1 arrest in bone marrow. 2BrIC inhibited the incorporation of BrdU into MPP (Lin−Kit+Sca1+ cells) relative to mice treated with formulation alone. See FIGS. 11A-11B. These data show that in vivo treatment with potent and selective CDK4/6 inhibitors induces potent PQ in early HSPC with more modest effects in more differentiated proliferating hematologic cells.

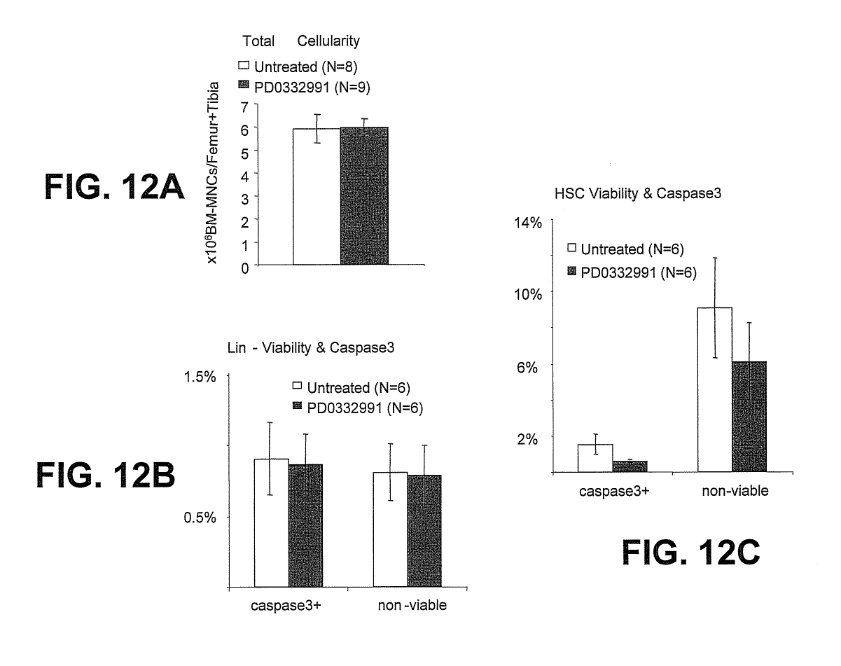

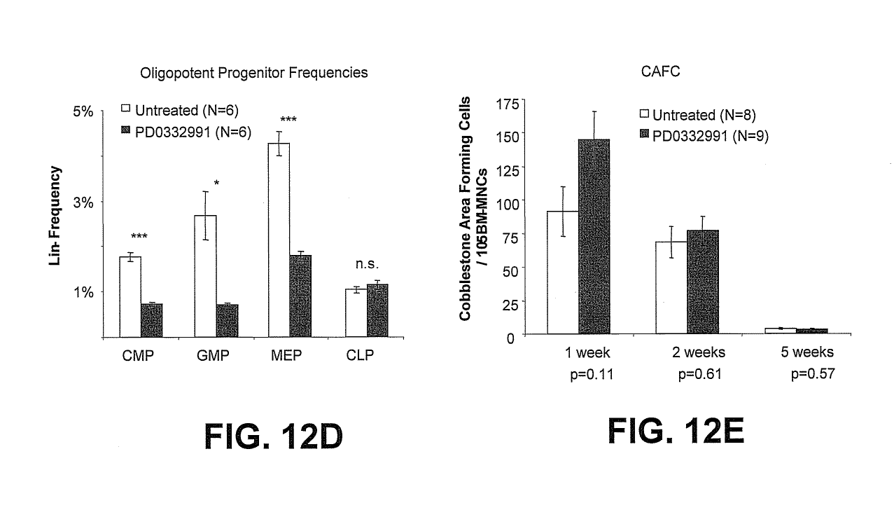

The effects on immunophenotypic HSPC frequency were validated by other methods. Transient (48 hour) treatment with PD0332991 did not decrease total marrow cellularity (see FIG. 12A), but did decrease the absolute number of lineage negative cells (see FIG. 10D) without altering Lin− or HSC apoptosis or viability. See FIGS. 12B-12C. The frequency of the more abundant oligopotent progenitors declined (Lin−cKit+Sca1−; see FIGS. 10D and 12D), with an associated relative increase in HSC and MPP frequencies. Cobblestone area forming cell assays confirmed that transient CDK4/6 inhibition did not decrease in vivo HSPC number. See FIG. 12E. In combination, these data suggest a gradient of dependence on CDK4/6 activity for proliferation during myeloid/erythroid differentiation: the least differentiated cells (HSC, MPP and CMP) appear to be the most dependent, with more differentiated elements (GMP and MEP) being less dependent, and even more differentiated myeloid and erythroid cells proliferating independently of CDK4/6 activity.

### Example 5

### Enhanced Survival in IR-Treated Animals

Adult female C57Bl/6 mice were exposed to 6.5, 7.5, or 8.5 Gy with or without PD0332991 treatment and followed for 40 days post-TBI. See FIGS. 13A-13F and 14A-14C. The LD₉₀ for IR was determined to be about 7.5 Gy. Marked protection from the hematologic toxicity of peri-lethal doses of TBI was observed: nearly all untreated mice succumbed to death from hematologic toxicity when exposed to 7.5 Gy, whereas all treated mice survived. See FIGS. 13B-13F. Treatment with the selective CDK4/6 inhibitor afforded a similar degree of radioprotection in two other inbred strains (C3H and FVB/n). See FIGS. 14A for C3H and FIG. 2E for FVB/n. Compared to untreated mice after 8.5 Gy TBI, a single dose of PD0332991 four hours prior to IR treatment significantly increased the term survival (13% vs. 0%) and prolonged the median survival (19 days vs. 13 days). See FIG. 14C. All mice survived after a dose of 6.5 Gy regardless of PD0332991 treatment. See FIG. 148. In accord with the in vitro results, mice administered less-selective CDK inhibitors showed no survival benefit after lethal TBI. It appears that PQ resulting from transient CDK4/6 inhibition around the time of TBI enhances radioresistance in vivo.

More particularly, animals were given a CDK4/6 inhibitor at different times relative to TBI at the LD₉₀. See FIGS. 13A-13F. As radioprotection was observed when animals were treated with two doses pre- and one dose post-TBI (see FIGS. 13A-13B), further studies were undertaken to determine which of these doses was most important for radioprotection. Without being bound to any one theory, it appeared that most of the benefit of the treatment schedule was associated PD 0332991 treatment immediately prior to or contemporaneous with TBI. Mice treated with a single −4 hour dose or a single time 0 dose demonstrated survival similar to animals treated on the multi-dose (−28, −4, +20) schedule. See FIGS. 13B-13E. However, even the survival of mice treated with a single dose 20 hours after TBI was significantly enhanced. See FIG. 13F. As therapeutic serum levels are not achieved until >30 minutes after gavage and then persist for 10-20 hours, these observations suggest that a period of PQ lasting for several (>20) hours after the induction of DNA damage is beneficial. Although there are a few known compounds that protect from radiotoxicity when administered prior to IR (i.e. “radioprotectants”) (see Burdelya et al., Science, 320, 226-230 (2008); and Weiss and Landauer, Int J. Radiat. Biol., 85, 539-573 (2009)), previous to the presently disclosed subject matter, no known hematologic “radiomitigants” (i.e., compounds that reduce hematotoxicity when administered many hours after an exposure to TBI) are believed to have been reported.

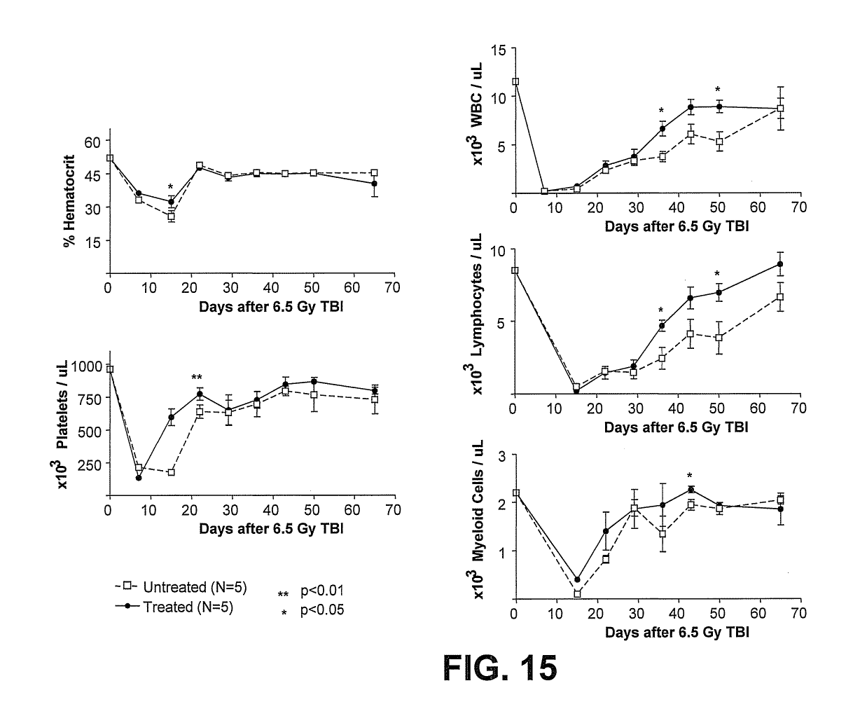

The peripheral blood of animals after IR exposure was studied to confirm that the improved survival was due to protection of hematopoietic lineages. As reported by others (see Na Nakorn et al., J. Clin. Invest., 109, 1579-1585 (2002), lethality from high-dose TBI was associated with morbid anemia and thrombocytopenia. This was significantly ameliorated by PD0332991 treatment in lethally irradiated mice. See FIG. 13G. Additionally, PD0332991 diminished count nadirs and caused more rapid count recovery after a sub-lethal dose of TBI. See FIG. 15. Importantly, PQ therapy had a beneficial effect on the recovery of all peripheral blood lineages: platelets, erythrocytes, myeloid cells (granulocytes+monocytes), and peripheral lymphocytes. The improvement in quadrilineage hematopoiesis after TBI is consistent with the notion that CDK4/6 inhibition exerts maximal radioprotection in the early HSPCs rendered quiescent by CDK4/6 inhibitor treatment.

When followed 210-274 days post-TBI, no deaths were seen in any animals after 6.5 Gy TBI regardless of PD0332991 treatment. Only two of 18 mice (one C3H and one C57Bl/6) survived 7.5 Gy TBI in the absence of PQ, and these animals showed no evidence of disease 143 to 252 days post-TBI. Of 29 mice surviving the acute toxicity of 7.5 Gy TBI in the setting of PQ, there was one death of unknown cause at day 99 post-TBI, with the remaining mice disease free 101-251 days post-TBI. Blood counts on long-term surviving animals were comparable among unirradiated and irradiated mice, with or without PD0332991 treatment at the time of TBI. See FIG. 16. No evidence of myeloproliferative disorder or myelodysplasia was seen in animals of these long-term surviving cohorts. These data indicate that PQ does not exacerbate the late hematologic toxicity associated with sub-lethal TBI, and affords good long-term hematologic radioprotection even after a lethal dose of TBI.

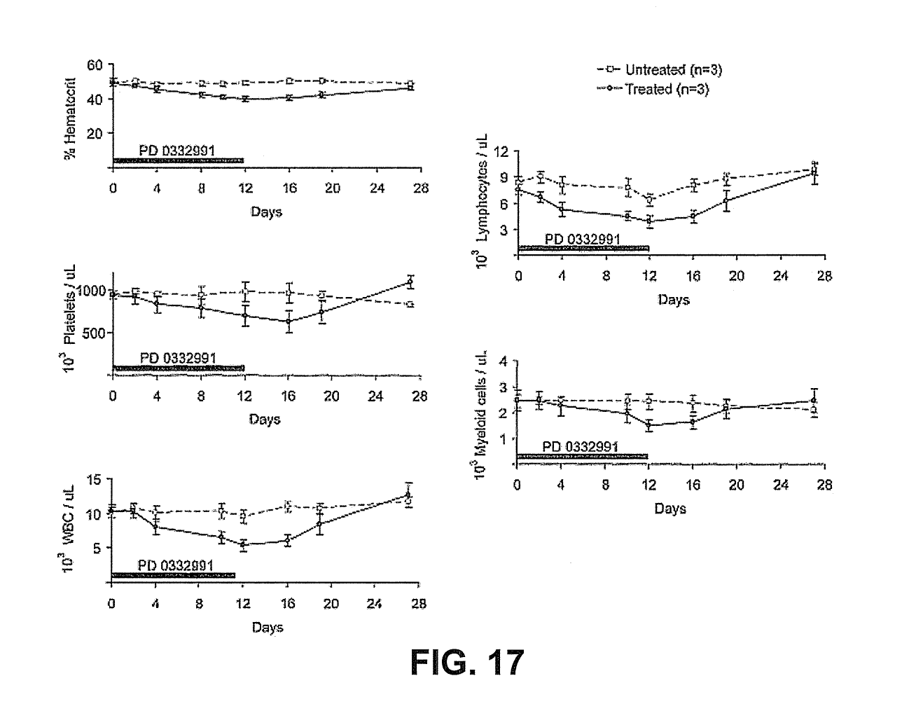

In accord with this model, serial daily treatment with PD0332991 for 12 days caused a modest decrease in the erythroid, platelet and myeloid (monocyte+granulocyte) lineages which only became apparent after 8 days of treatment, and which began to improve within 4 days upon cessation of PD0332991. See FIG. 17. These observations are consistent with the kinetics and degree of myelosuppression seen when tumor bearing mice (see Ramsey et al., Cancer Res., 67, 4732-4741 (2007); and Fry et al., Mol. Cancer Ther., 3, 1427-1438 (2004)) and human patients with malignancies (see O'Dwyer et al., “A Phase I does escalation trial of a daily oral CDK4/6 Inhibitor PD 0332991” in American Society of Clinical Oncology (ASCO, Chicago, Ill., 2007)) are serially treated with PD0332991. The noted decreases might be expected to enhance the adverse effects of radiation therapy. However, unexpectedly as shown herein, hematopoietic cells are protected from adverse effects. Further, these data confirm that the short-term, proliferative production of differentiated effector cells of the peripheral blood is relatively resistant to CDK4/6 inhibition, and that the myelosuppressive effects of CDK4/6 inhibition are rapidly reversible in vivo.

Accordingly, the presently disclosed data suggest that selective pharmacologic inhibition of CDK4/6 protects CDK4/6-dependent cells from IR both in vivo and in vitro by inducing G1-arrest. Of note, PQ is protective in vivo even when initiated well after exposure to IR. See FIG. 13F. Without being bound to any one theory, the data suggests that CDK4/6 inhibition protects early hematopoietic progenitors by lengthening the period of G1/0 in cells harboring unrepaired DNA damage for several hours after TBI. Implicit in this view is the assumption that attempted G1-S traversal in the setting of unrepaired DNA damage is a particularly toxic event, consistent with the increased radiosensitivity noted in late G1 and early S phases. See Sinclair and Morton, Radiation Research, 29, 450-474 (1966); and Terasima and Tolmach, Science, 140, 490-492 (1963).

Current interventions to mitigate radiation toxicity have relied on a combination of supportive care, growth factors, cytokines, and specific chelating agents, none of which are effective when administered well after radiation exposure. See Weiss and Landauer, Int, J. Radiat. Biol., 85, 539-573 (2009). While growth factor support with agents such as G/GM-CSF or erythropoietin has been shown to attenuate the toxic effects of DNA damaging agents (see Herodin et al., Blood, 101, 2609-2616 (2003); and Uckun et al., Blood, 75, 638-645 (1990)) the small molecule PQ approach appears to have greater magnitude of effect, a longer effective duration after exposure, and protects without the toxicities of these biologics. Moreover, PQ ameliorates DNA damage induced thrombocytopenia (see FIG. 13G), a significant unmet need in clinical oncology and radiation mitigation. While late hematologic toxicity has been associated with growth factor support after exposure to DNA damaging agents in both humans (see Hershman et al., J. Nat. Cancer Inst., 99, 196-205 (2007) and Le Deley et al., J. Clin. Oncol., 25, 292-300 (2007)) and mice (see Herodin et al., Blood, 101, 2609-2616 (2003)), PQ does not appear to augment late hematologic toxicity after TBI. See FIG. 16. Furthermore, growth factor support and PQ appear to enhance count recovery through different mechanisms: the former by inhibiting apoptosis, increasing HSPC proliferation and modulating lineage choice; whereas the latter by enhancing DNA repair. See FIGS. 4A-4B.

### Example 6

### Synthesis of PD

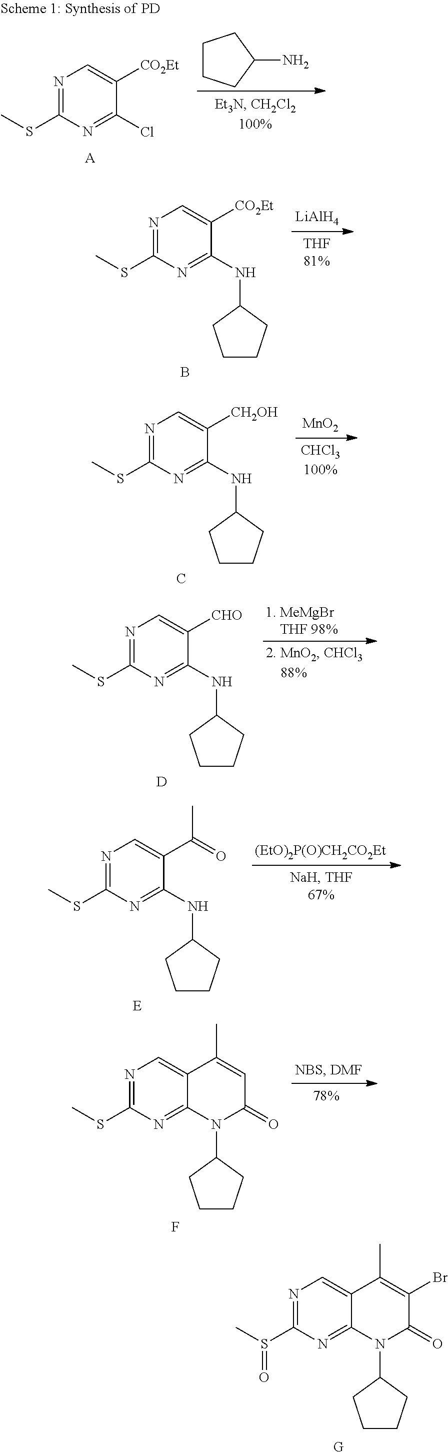

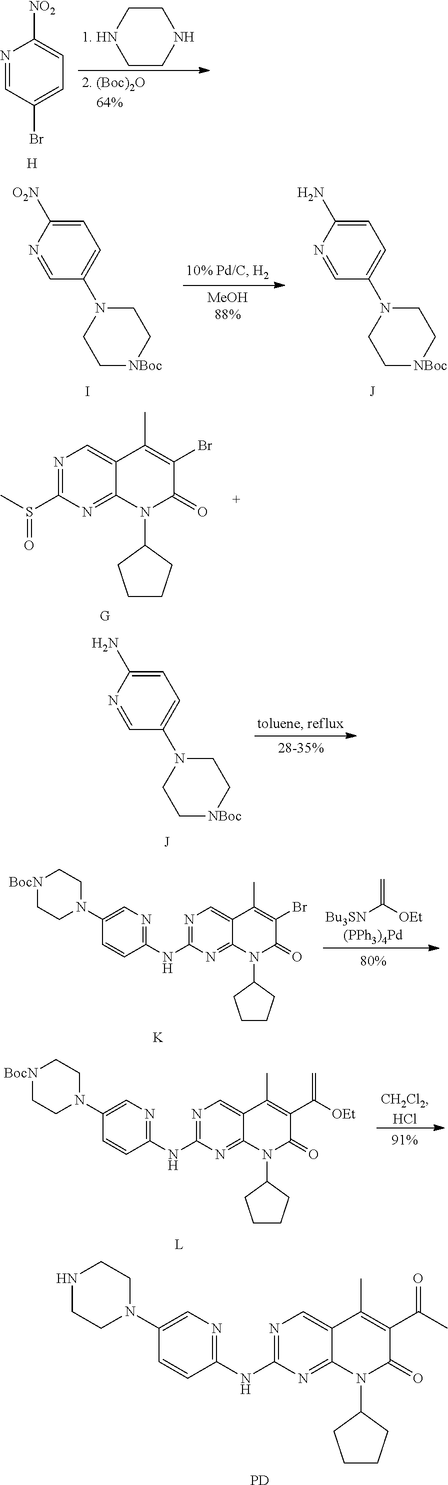

PD was synthesized as shown above in Scheme 1. Reactions shown in Scheme 1 generally followed previously reported procedures (see VandelWel et al., J. Med Chem., 48, 2371-2387 (2005); and Toogood et al., J. Med. Chem., 48, 2388-2406 (2005)), with the exceptions of the reaction converting compound D to compound E and the reaction converting compound F to compound G.

Conversion of Compound D to Compound E:

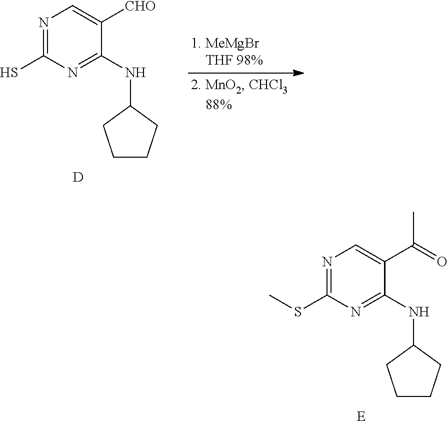

Compound D (40 g, 169 mmol) was dissolved in anhydrous THF (800 mL) under nitrogen and the solution was cooled in ice bath, to which MeMgBr was added slowly (160 mL, 480 mmol, 3 M in ether) and stirred for 1 h. The reaction was quenched with saturated aqueous NH₄Cl the partitioned between water and EtOAc. The organic layer was separated and the aqueous layer was extracted with EtOAc. The combined organic were washed with brine and dried over MgSO₄. Concentration gave an intermediate product as an oil (41.9 g, 98%).

The above intermediate (40 g, 158 mmol) was dissolved in dry CHCl₃ (700 mL). MnO₂ (96 g, 1.11 mol) was added and the mixture was heated to reflux with stirring for 18 h and another MnO₂ (34 g, 395 mmol) was added and continue to reflux for 4 h. The solid was filtrated through a Celite pad and washed with CHCl₃. The filtrate was concentrated to give a yellow solid compound E (35 g, 88%), Mp: 75.8-76.6° C.

Conversion of Compound F to Compound G:

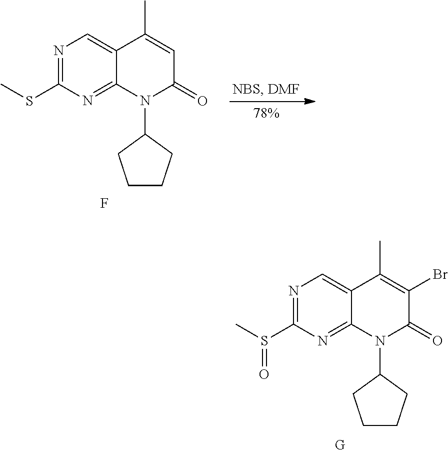

Compound F (5 g, 18.2 mmol) was dissolved in anhydrous DMF (150 mL) and NBS (11.3 g, 63.6 mmol) was added. The reaction mixture was stirred at r.t. for 3.5 h and then poured into H₂O (500 mL), the precipitate was filtered and washed with H₂O. The solid recrystallized from EtOH to give compound G as a white solid (5.42 g, 80.7%), mp: 210.6-211.3° C.

Characterization Data for PD:

LC-MS: 448.5 (ESI, M+H). Purity: ˜99%

¹H NMR(300 MHz, D₂O): 9.00(s, 1H), 8.12 (dd, J=9.3 Hz, 2.1 Hz, 1H), 7.81(d, J=2.4 Hz, 1H), 7.46(d, J=9.6 Hz, 1H), 5.80-5.74 (m, 1H), 3.57-3.48(m, 8H), 2.48(s, 3H), 2.37(s, 3H), 2.13-1.94(m, 6H), 1.73-1.71(m, 2H).

¹³C NMR (75 MHz, D₂O): 203.6, 159.0, 153.5, 153.3, 152.2, 139.9, 139.4, 139.2, 133.1, 129.0, 118.7, 113.8, 107.4, 51.8, 42.2, 40.0, 28.0, 25.2, 22.6, 10.8.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of reducing or preventing the effects of ionizing radiation on healthy cells in a subject who has been exposed to, will be exposed to, or is at risk of incurring exposure to ionizing radiation, wherein said healthy cells are hematopoietic stem cells or hematopoietic progenitor cells, the method comprising administering to the subject an effective amount of an inhibitor compound, or a pharmaceutically acceptable form thereof, wherein the inhibitor compound selectively inhibits cyclin-dependent kinase 4 (CDK4) and/or cyclin-dependent kinase 6 (CDK6).

2. The method of claim 1, wherein the inhibitor compound is selected from the group consisting of a pyrido[2,3-d]pyrimidine, a triaminopyrimidine, an aryl[a]pyrrolo[3,4-c]carbazole, a nitrogen-containing heteroaryl-substituted urea, a 5-pyrimidinyl-2-aminothiazole, a benzothiadiazine, and an acridinethione.

3. The method of claim 2, wherein the pyrido[2,3-d]pyrimidine is a pyrido[2,3-d]pyrimidin-7-one or a 2-amino-6-cyano-pyrido[2,3-d]pyrimidin-4-one.

4. The method of claim 3, wherein the pyrido[2,3-d]pyrimidin-7-one is a 2-(2′-pyridyl)amino pyrido[2,3-d]pyrimidin-7-one.

5. The method of claim 4, wherein the pyrido[2,3-d]pyrimidin-7-one is 6-acetyl-8-cyclopentyl-5-methyl-2-(5-piperazin-1-yl-pyridin-2-ylamino)-8H-pyrido-[2,3-d]pyrimidin-7-one.

6. The method of claim 2, wherein the aryl[a]pyrrolo[3,4-c]carbazole is selected from the group consisting of a napthyl[a]pyrrolo[3,4-c]carbazole, an indolo[a]pyrrolo[3,4-c]carbazole, a quinolinyl[a]pyrrolo[3,4-c]carbazole, and an isoquinolinyl[a]pyrrolo[3,4-c]carbazole.

7. The method of claim 6, wherein the aryl[a]pyrrolo[3,4-c]carbazole is 2-bromo-12,13-dihydro-5H-indolo[2,3-a]pyrrolo[3,4]-carbazole-5,6-dione.

8. The method of claim 1, wherein the inhibitor compound selectively inhibits both cyclin-dependent kinase 4 (CDK4) and cyclin-dependent kinase 6 (CDK6).

9. The method of claim 1, wherein the inhibitor compound is a non-naturally occurring compound.

10. The method of claim 1, wherein the inhibitor compound selectively induces G1 arrest in cyclin-dependent kinase 4 (CDK4)- and/or cyclin-dependent kinase 6 (CDK6)-dependent cells.

11. The method of claim 10, wherein the inhibitor compound induces substantially pure G1 arrest in cyclin-dependent kinase 4 (CDK4)- and/or cyclin-dependent kinase 6 (CDK6)-dependent cells.

12. The method of claim 1, wherein the inhibitor compound is substantially free of off-target effects.

13. The method of claim 12, wherein the off-target effects are one or more of the group consisting of long term toxicity, anti-oxidant effects, estrogenic effects, tyrosine kinase inhibition, inhibition of cyclin-dependent kinases (CDKs) other than cyclin-dependent kinase 4/6 (CDK4/6), and cell cycle arrest in CDK4/6-independent cells.

14. The method of claim 1, wherein the subject is a mammal.

15. The method of claim 1, wherein the inhibitor compound is administered to the subject by one of the group consisting of oral administration, topical administration, intranasal administration, inhalation, and intravenous administration.

16. The method of claim 1, wherein the inhibitor compound is administered to the subject prior to exposure to the ionizing radiation, during exposure to the ionizing radiation, after exposure to the ionizing radiation, or a combination thereof.

17. The method of claim 16, wherein the inhibitor compound is administered to the subject less than about 24 hours prior to exposure to the ionizing radiation.

18. The method of claim 16, wherein the inhibitor compound is administered to the subject prior to exposure to the ionizing radiation such that the compound reaches peak serum levels during exposure to the ionizing radiation.

19. The method of claim 18, wherein the inhibitor compound is 6-acetyl-8-cyclopentyl-5-methyl-2-(5-piperazin-1-yl-pyridin-2-ylamino)-8H-pyrido-[2,3-d]pyrimidin-7-one and wherein said inhibitor compound is administered orally to the subject 4 hours prior to exposure to the ionizing radiation.

20. The method of claim 16, wherein the inhibitor compound is administered to the subject after exposure to the ionizing radiation.

21. The method of claim 20, wherein the inhibitor compound is administered to the subject about 24 hours or more after exposure to the ionizing radiation.

22. The method of claim 1, wherein the healthy cells are selected from the group consisting of long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs).

23. The method of claim 1, wherein administration of the inhibitor compound provides temporary pharmacologic quiescence of hematopoietic stem and/or progenitor cells in the subject.

24. The method of claim 1, wherein the subject has incurred ionizing radiation or is at risk of incurring exposure to ionizing radiation as the result of radiological agent exposure during warfare, a radiological terrorist attack, an industrial accident, other occupational exposure or space travel.

25. The method of claim 1, wherein the subject is undergoing radio-therapy to treat a disease.

26. The method of claim 25, wherein administration of the inhibitor compound does not affect growth of diseased cells.

27. The method of claim 25, wherein the disease is cancer.

28. The method of claim 27, wherein the cancer is characterized by one or more of the group consisting of increased activity of cyclin-dependent kinase 1 (CDK1), increased activity of cyclin-dependent kinase 2 (CDK2), loss or absence of retinoblastoma tumor suppressor protein (RB), high levels of MYC expression, increased cyclin E and increased cyclin A.

29. The method of claim 25, wherein administration of the inhibitor compound allows for a higher dose of ionizing radiation to be used to treat the disease than the dose that would be used in the absence of administration of the inhibitor compound.

30. The method of claim 1, wherein the method is free of long-term hematologic toxicity.

31. The method of claim 1, wherein administration of the inhibitor compound results in reduced anemia, reduced lymphopenia, reduced thrombocytopenia, or reduced neutropenia compared to that expected after exposure to ionizing radiation in the absence of administration of the inhibitor compound.
